SEQUENCE LISTING (40 sequences)

SEQ ID NO: 1 - protein, 120 aa, synthetic construct
EVQLVESGGGLVKPGGSLRLSCAASGFTFSSYHMSWVRQAPGKCLELVSAINSNGINTYYLDTVKGRFTISRDNAKNSLYLQMNSLRAEDTAVYYCARHEDYYGFAMDYWGQGTLVTVSS

SEQ ID NO: 2 - protein, 107 aa, synthetic construct
DIVMTQSPDSLAVSLGERATINCKASQSVSNDVAWYQQKPGQPPKLLIYYVSHRFTGVPDRFSGSGSGTDFTLTISSLQAEDVAVYFCHQAYRSPWTFGCGTKLEIK

SEQ ID NO: 3 - protein, 5 aa, synthetic construct
SYHMS

SEQ ID NO: 4 - protein, 17 aa, synthetic construct
AINSNGINTYYLDTVKG

SEQ ID NO: 5 - protein, 11 aa, synthetic construct
HEDYYGFAMDY

SEQ ID NO: 6 - protein, 11 aa, synthetic construct
KASQSVSNDVA

SEQ ID NO: 7 - protein, 7 aa, synthetic construct
YVSHRFT

SEQ ID NO: 8 - protein, 9 aa, synthetic construct
HQAYRSPWT

SEQ ID NO: 9 - protein, 119 aa, synthetic construct
QVQLVQSGAEVKKPGASVKVSCKTSGYAFTEYTMHWVRQAPGKGLEWMGGINPNTGGTTYNQKFQGRVTLTVDKSSSTAYMELSSLRSEDTVVYYCAKLLRLMYYFDYWGQGTLVTVSS

SEQ ID NO: 10 - protein, 107 aa, synthetic construct
DIQMTQSPSSLSASVGDRVTITCQASQDVRTAVAWYQQKPGKAPKLLIYSASYRYTGVPSRFSGSGSGTDFTFTISSLQPEDIATYYCHQHYITPWTFGGGTKVEIK

SEQ ID NO: 11 - protein, 5 aa, synthetic construct
EYTMH

SEQ ID NO: 12 - protein, 17 aa, synthetic construct
GINPNTGGTTYNQKFQG

SEQ ID NO: 13 - protein, 10 aa, synthetic construct
LLRLMYYFDY

SEQ ID NO: 14 - protein, 11 aa, synthetic construct
QASQDVRTAVA

SEQ ID NO: 15 - protein, 7 aa, synthetic construct
SASYRYT

SEQ ID NO: 16 - protein, 9 aa, synthetic construct
HQHYITPWT

SEQ ID NO: 17 - protein, 446 aa, synthetic construct
QVQLVQSGAEVKKPGASVKVSCKTSGYAFTEYTMHWVRQAPGKGLEWMGGINPNTGGTTYNQKFQGRVTLTVDKSSSTAYMELSSLRSEDTVVYYCAKLLRLMYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 214 aa, synthetic construct
DIQMTQSPSSLSASVGDRVTITCQASQDVRTAVAWYQQKPGKAPKLLIYSASYRYTGVPSRFSGSGSGTDFTFTISSLQPEDIATYYCHQHYITPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 486 aa, synthetic construct
DIVMTQSPDSLAVSLGERATINCKASQSVSNDVAWYQQKPGQPPKLLIYYVSHRFTGVPDRFSGSGSGTDFTLTISSLQAEDVAVYFCHQAYRSPWTFGCGTKLEIKGGGGSGGGGSGGGGSGGGGSEVQLVESGGGLVKPGGSLRLSCAASGFTFSSYHMSWVRQAPGKCLELVSAINSNGINTYYLDTVKGRFTISRDNAKNSLYLQMNSLRAEDTAVYYCARHEDYYGFAMDYWGQGTLVTVSSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 20 - protein, 449 aa, synthetic construct
QVQLVQSGAEVKKPGASVKVSCKTSGYAFTEYTMHWVRQAPGKGLEWMGGINPNTGGTTYNQKFQGRVTLTVDKSSSTAYMELSSLRSEDTVVYYCAKLLRLMYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 484 aa, synthetic construct
DIVMTQSPDSLAVSLGERATINCKASQSVSNDVAWYQQKPGQPPKLLIYYVSHRFTGVPDRFSGSGSGTDFTLTISSLQAEDVAVYFCHQAYRSPWTFGCGTKLEIKGGGGSGGGGSGGGGSGGGGSEVQLVESGGGLVKPGGSLRLSCAASGFTFSSYHMSWVRQAPGKCLELVSAINSNGINTYYLDTVKGRFTISRDNAKNSLYLQMNSLRAEDTAVYYCARHEDYYGFAMDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 22 - protein, 694 aa, synthetic construct
DIQMTQSPSSLSASVGDRVTITCQASQDVRTAVAWYQQKPGKAPKLLIYSASYRYTGVPSRFSGSGSGTDFTFTISSLQPEDIATYYCHQHYITPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGQVQLVQSGAEVKKPGASVKVSCKTSGYAFTEYTMHWVRQAPGKGLEWMGGINPNTGGTTYNQKFQGRVTLTVDKSSSTAYMELSSLRSEDTVVYYCAKLLRLMYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 695 aa, synthetic construct
DIVMTQSPDSLAVSLGERATINCKASQSVSNDVAWYQQKPGQPPKLLIYYVSHRFTGVPDRFSGSGSGTDFTLTISSLQAEDVAVYFCHQAYRSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGEVQLVESGGGLVKPGGSLRLSCAASGFTFSSYHMSWVRQAPGKGLELVSAINSNGINTYYLDTVKGRFTISRDNAKNSLYLQMNSLRAEDTAVYYCARHEDYYGFAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 24 - protein, 697 aa, synthetic construct
DIQMTQSPSSLSASVGDRVTITCQASQDVRTAVAWYQQKPGKAPKLLIYSASYRYTGVPSRFSGSGSGTDFTFTISSLQPEDIATYYCHQHYITPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGQVQLVQSGAEVKKPGASVKVSCKTSGYAFTEYTMHWVRQAPGKGLEWMGGINPNTGGTTYNQKFQGRVTLTVDKSSSTAYMELSSLRSEDTVVYYCAKLLRLMYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 698 aa, synthetic construct
DIVMTQSPDSLAVSLGERATINCKASQSVSNDVAWYQQKPGQPPKLLIYYVSHRFTGVPDRFSGSGSGTDFTLTISSLQAEDVAVYFCHQAYRSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGEVQLVESGGGLVKPGGSLRLSCAASGFTFSSYHMSWVRQAPGKGLELVSAINSNGINTYYLDTVKGRFTISRDNAKNSLYLQMNSLRAEDTAVYYCARHEDYYGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 26 - protein, 701 aa, synthetic construct
QVQLVQSGAEVKKPGASVKVSCKTSGYAFTEYTMHWVRQAPGKGLEWMGGINPNTGGTTYNQKFQGRVTLTVDKSSSTAYMELSSLRSEDTVVYYCAKLLRLMYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSDIVMTQSPDSLAVSLGERATINCKASQSVSNDVAWYQQKPGQPPKLLIYYVSHRFTGVPDRFSGSGSGTDFTLTISSLQAEDVAVYFCHQAYRSPWTFGCGTKLEIKGGGGSGGGGSGGGGSGGGGSEVQLVESGGGLVKPGGSLRLSCAASGFTFSSYHMSWVRQAPGKCLELVSAINSNGINTYYLDTVKGRFTISRDNAKNSLYLQMNSLRAEDTAVYYCARHEDYYGFAMDYWGQGTLVTVSS

SEQ ID NO: 27 - protein, 704 aa, synthetic construct
QVQLVQSGAEVKKPGASVKVSCKTSGYAFTEYTMHWVRQAPGKGLEWMGGINPNTGGTTYNQKFQGRVTLTVDKSSSTAYMELSSLRSEDTVVYYCAKLLRLMYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSDIVMTQSPDSLAVSLGERATINCKASQSVSNDVAWYQQKPGQPPKLLIYYVSHRFTGVPDRFSGSGSGTDFTLTISSLQAEDVAVYFCHQAYRSPWTFGCGTKLEIKGGGGSGGGGSGGGGSGGGGSEVQLVESGGGLVKPGGSLRLSCAASGFTFSSYHMSWVRQAPGKCLELVSAINSNGINTYYLDTVKGRFTISRDNAKNSLYLQMNSLRAEDTAVYYCARHEDYYGFAMDYWGQGTLVTVSS

SEQ ID NO: 28 - protein, 703 aa, synthetic construct
DIVMTQSPDSLAVSLGERATINCKASQSVSNDVAWYQQKPGQPPKLLIYYVSHRFTGVPDRFSGSGSGTDFTLTISSLQAEDVAVYFCHQAYRSPWTFGCGTKLEIKGGGGSGGGGSGGGGSGGGGSEVQLVESGGGLVKPGGSLRLSCAASGFTFSSYHMSWVRQAPGKCLELVSAINSNGINTYYLDTVKGRFTISRDNAKNSLYLQMNSLRAEDTAVYYCARHEDYYGFAMDYWGQGTLVTVSSGGGGSGGGGSQVQLVQSGAEVKKPGASVKVSCKTSGYAFTEYTMHWVRQAPGKGLEWMGGINPNTGGTTYNQKFQGRVTLTVDKSSSTAYMELSSLRSEDTVVYYCAKLLRLMYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 706 aa, synthetic construct
DIVMTQSPDSLAVSLGERATINCKASQSVSNDVAWYQQKPGQPPKLLIYYVSHRFTGVPDRFSGSGSGTDFTLTISSLQAEDVAVYFCHQAYRSPWTFGCGTKLEIKGGGGSGGGGSGGGGSGGGGSEVQLVESGGGLVKPGGSLRLSCAASGFTFSSYHMSWVRQAPGKCLELVSAINSNGINTYYLDTVKGRFTISRDNAKNSLYLQMNSLRAEDTAVYYCARHEDYYGFAMDYWGQGTLVTVSSGGGGSGGGGSQVQLVQSGAEVKKPGASVKVSCKTSGYAFTEYTMHWVRQAPGKGLEWMGGINPNTGGTTYNQKFQGRVTLTVDKSSSTAYMELSSLRSEDTVVYYCAKLLRLMYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 120 aa, synthetic construct
EVQLVESGGGLVKPGGSLRLSCAASGFTFSSYHMSWVRQAPGKGLELVSAINSNGINTYYLDTVKGRFTISRDNAKNSLYLQMNSLRAEDTAVYYCARHEDYYGFAMDYWGQGTLVTVSS

SEQ ID NO: 31 - protein, 107 aa, synthetic construct
DIVMTQSPDSLAVSLGERATINCKASQSVSNDVAWYQQKPGQPPKLLIYYVSHRFTGVPDRFSGSGSGTDFTLTISSLQAEDVAVYFCHQAYRSPWTFGQGTKLEIK

SEQ ID NO: 32 - protein, 214 aa, synthetic construct
DIVMTQSPDSLAVSLGERATINCKASQSVSNDVAWYQQKPGQPPKLLIYYVSHRFTGVPDRFSGSGSGTDFTLTISSLQAEDVAVYFCHQAYRSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 447 aa, synthetic construct
EVQLVESGGGLVKPGGSLRLSCAASGFTFSSYHMSWVRQAPGKGLELVSAISRDGDSTYYPDTVKGRFTISRDNAKNSLYLQMNSLRAEDTAVYYCSRHEDYFGFAMDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 450 aa, synthetic construct
EVQLVESGGGLVKPGGSLRLSCAASGFTFSSYHMSWVRQAPGKGLELVSAISRDGDSTYYPDTVKGRFTISRDNAKNSLYLQMNSLRAEDTAVYYCSRHEDYFGFAMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 214 aa, synthetic construct; identical to SEQ ID NO: 32

SEQ ID NO: 36 - protein, 447 aa, synthetic construct
EVQLVESGGGLVKPGGSLRLSCAASGFTFSSYHMSWVRQAPGKGLELVSAINSNGINTYYLDTVKGRFTISRDNAKNSLYLQMNSLRAEDTAVYYCARHEDYYGFAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 450 aa, synthetic construct
EVQLVESGGGLVKPGGSLRLSCAASGFTFSSYHMSWVRQAPGKGLELVSAINSNGINTYYLDTVKGRFTISRDNAKNSLYLQMNSLRAEDTAVYYCARHEDYYGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, synthetic construct
DIQMTQSPSSLSASVGDRVTITCKASQSVSNDVAWYQQKPGKAPKLLIYYVSNRFTGVPSRFSGSGSGTDFTLTISSLQPEDFATYFCQQAYRSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 447 aa, synthetic construct
EVQLVESGGGLVQPGGSLRLSCAASGFTFSSYHMSWIRQAPGKGLELVAAINSNGINTYYLDTVKGRFTISRDNAKNSLYLQMNSLRAEDTAVYYCARHEDYYGFAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 450 aa, synthetic construct
EVQLVESGGGLVQPGGSLRLSCAASGFTFSSYHMSWIRQAPGKGLELVAAINSNGINTYYLDTVKGRFTISRDNAKNSLYLQMNSLRAEDTAVYYCARHEDYYGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0117043 A1
(43) Pub. Date: Apr. 11, 2024

(54) BISPECIFIC ANTIBODY TARGETING CD112R AND TIGIT AND USE THEREOF

(71) Applicants: SHANGHAI JUNSHI BIOSCIENCES CO., LTD., SHANGHAI (CN); SUZHOU JUNMENG BIOSCIENCES CO., LTD., SUZHOU (CN)

(72) Inventors: Dandan LIU, SHANGHAI (CN); Jinwei ZHOU, SHANGHAI (CN); Yuehua ZHOU, SHANGHAI (CN); Jing ZHANG, SHANGHAI (CN); Sheng YAO, SHANGHAI (CN); Hui FENG, SHANGHAI (CN); Hui LIU, SHANGHAI (CN); Hongchuan LIU, SHANGHAI (CN); Li LI, SHANGHAI (CN); Qiang ZHAO, SHANGHAI (CN)

(73) Assignees: SHANGHAI JUNSHI BIOSCIENCES CO., LTD., SHANGHAI (CN); SUZHOU JUNMENG BIOSCIENCES CO., LTD., SUZHOU (CN)

(21) Appl. No.: 18/365,230

(22) Filed: Aug. 4, 2023

(30) Foreign Application Priority Data
Aug. 5, 2022 (CN) 202210937822.7

(51) Int. Cl. C07K 16/28; A61P 35/00

(52) U.S. Cl. CPC C07K 16/2803; A61P 35/00; C07K 2317/31

(57) ABSTRACT

The present disclosure provides a bispecific antibody including a binding domain that binds to CD112R and a binding domain that binds to TIGIT, and the binding domain that binds to CD112R includes: HCDR1, HCDR2 and HCDR3 of the amino acid sequence set forth in SEQ ID NO: 1, and/or LCDR1, LCDR2 and LCDR3 of the amino acid sequence set forth in SEQ ID NO: 2; and the HCDR1, HCDR2, HCDR3, LCDR1, LCDR2 and LCDR3 are defined according to the Kabat, IMGT, Chothia, AbM or Contact numbering system. The present disclosure further provides a polynucleotide encoding the antibody, an expression vector, a host cell and a method for expressing and purifying the antibody, a pharmaceutical composition including the antibody of the present disclosure, and use of the bispecific antibody for treating cancer.

### FIELD

The present disclosure relates to the field of antibody drugs and particularly to a bispecific antibody targeting CD112R and TIGIT, a preparation method therefor, a pharmaceutical composition including the antibody, and use thereof.

### REFERENCE TO SEQUENCE LISTING

A Sequence Listing conforming to the rules of USPTO Standard is hereby incorporated by reference. Said Sequence Listing has been filed as an electronic document via PatentCenter in ASCII format encoded as XML. The electronic document, created on Aug. 1, 2023, is entitled “225136 1CNUS.xml”, and is 46,961 bytes in size.

### BACKGROUND

The immune system is a highly complex system composed of many cell types, including but not limited to T cells, B cells, natural killer cells, antigen-presenting cells, dendritic cells, monocytes, and macrophages. These cells possess complex and subtle systems to control their interactions and responses. Cells utilize activation and inhibition mechanisms and feedback loops to maintain control of the response and do not allow the negative consequences of an uncontrolled immune response (e.g., autoimmune disease).

CD112R (PVRIG), also known as poliovirus receptor-associated immunoglobulin domain-containing protein, Q6DKI7, or C7orf15, is a transmembrane domain protein of 326 amino acids, including a signal peptide (amino acids 1 to 40), an extracellular domain (amino acids 41 to 171), a transmembrane domain (amino acids 172 to 190), and a cytoplasmic domain (amino acids 191 to 326). CD112R binds to poliovirus receptor-related 2 (PVLR2, also known as nectin-2, CD112, herpesvirus entry mediator B (HVEB), or human plasma membrane glycoprotein), the binding partner of CD112R.

The administration of anti-CD112R immunotherapy offers the opportunity to increase, enhance, and maintain the immune response. CD112R is an inhibitory receptor mainly expressed by T cells and NK cells and competes with the activation receptor CD226 for binding to CD112. CD112 has a higher affinity for the interaction with CD112R than that with CD226, and can thus effectively regulate CD226-mediated cell activation. Anti-CD112R antibodies that block the interaction of CD112R with CD112 directly limit downstream inhibitory signaling of CD112R and also promote immune cell activation to a greater extent by increasing the interaction of CD226 with CD112.

Treatment with anti-CD112R antibodies provides an opportunity to down-regulate inhibitory signals that may occur when CD112R-expressing immune cells bind to CD112 on tumor cells and/or myeloid cells within the tumor microenvironment, and may enhance, increase, and maintain the anti-tumor immune response.

Another immune checkpoint of interest is TIGIT (T cell immunoreceptor with Ig and ITIM domains), which is an immunoregulatory receptor consisting of an extracellular immunoglobulin domain, a type I transmembrane region and two ITIM motifs and expressed predominantly on activated T cells and NK cells (Stanietsky et al., PNAS. 2009, 106, 17858-17863). TIGIT recognizes ligands poliovirus receptor (PVR, CD155) and the Nectin 2 (PVRL2/CD112), which are overexpressed on a variety of different tumor cells. It has been reported that TIGIT binds to the ligand PVR with high affinity. The binding of TIGIT to the ligand will activate inhibitory signals mediated by the following two motifs present in the cytoplasmic tail of TIGIT: an immunoreceptor tail tyrosine (ITT)-like motif and an immunodominant tyrosine-based inhibitory (ITIM) motif (Liu et al., Cell death and differentiation 2013, 20, 456-464; Stanietsky et al., European journal of immunology, 2013, 43, 2138-2150). The tumor cell surface ligand, through its binding to the ITIM domain of TIGIT on the surfaces of NK cells and T cells, inhibits the cytotoxicity of the NK cells and the activity of the T cells, and to mediate the immune evasion mechanism of tumor cells.

There have not as yet been approved CD112R/TIGIT bispecific antibody drugs on the market. There remains a need to develop bispecific antibodies targeting CD112R and TIGIT with good stability and activity.

### SUMMARY

In one embodiment, the present disclosure provides a bispecific antibody including a binding domain that binds to CD112R and a binding domain that binds to TIGIT, and the binding domain that binds to CD112R includes:
HCDR1, HCDR2 and HCDR3 of the amino acid sequence set forth in SEQ ID NO: 1, and/or LCDR1, LCDR2 and LCDR3 of the amino acid sequence set forth in SEQ ID NO: 2;
and the HCDR1, HCDR2, HCDR3, LCDR1, LCDR2 and LCDR3 are defined according to the Kabat, IMGT, Chothia, AbM or Contact numbering system.

In some embodiments, the binding domain of the present disclosure that binds to TIGIT includes:
HCDR1, HCDR2 and HCDR3 of the amino acid sequence set forth in SEQ ID NO: 9, and/or LCDR1, LCDR2 and LCDR3 of the amino acid sequence set forth in SEQ ID NO: 10;
and the HCDR1, HCDR2, HCDR3, LCDR1, LCDR2 and LCDR3 are defined according to the Kabat, IMGT, Chothia, AbM or Contact numbering system.

In some embodiments, the binding domain of the present disclosure that binds to CD112R includes:
a heavy chain variable region having at least 90%, 91%, 92%, 93%, 94%, 95%, 96%, 97%, 98%, 99% or 100% sequence identity to the amino acid sequence set forth in SEQ ID NO: 1, and a light chain variable region having at least 90%, 91%, 92%, 93%, 94%, 95%, 96%, 97%, 98%, 99% or 100% sequence identity to the amino acid sequence set forth in SEQ ID NO: 2; or
a heavy chain variable region having at least 90%, 91%, 92%, 93%, 94%, 95%, 96%, 97%, 98%, 99% or 100% sequence identity to the amino acid sequence set forth in SEQ ID NO: 30, and a light chain variable region having at least 90%, 91%, 92%, 93%, 94%, 95%, 96%, 97%, 98%, 99% or 100% sequence identity to the amino acid sequence set forth in SEQ ID NO: 31.

In some embodiments, the binding domain of the present disclosure that binds to TIGIT includes: a heavy chain variable region having at least 90%, 91%, 92%, 93%, 94%, 95%, 96%, 97%, 98%, 99% or 100% sequence identity to the amino acid sequence set forth in SEQ ID NO: 9, and a light chain variable region having at least 90%, 91%, 92%, 93%, 94%, 95%, 96%, 97%, 98%, 99% or 100% sequence identity to the amino acid sequence set forth in SEQ ID NO: 10.

In some embodiments, the binding domain of the present disclosure that binds to CD112R is an ScFab fragment, a Fab fragment, an scFv fragment or an Fv fragment, and the binding domain that binds to TIGIT is an ScFab fragment, a Fab fragment, an scFv fragment or an Fv fragment.

In some embodiments, the bispecific antibody of the present disclosure further includes an Fc domain.

In some embodiments, the Fc domain of the present disclosure is an IgG Fc domain, such as an IgG1 Fc domain, an IgG2 Fc domain, an IgG3 Fc domain or an IgG4 Fc domain.

In some embodiments, the bispecific antibody of the present disclosure is a bivalent or tetravalent bispecific antibody; In one embodiment, the binding domain that binds to TIGIT is linked to the N-terminus of the Fc domain by a hinge region, and the binding domain that binds to CD112R is linked to the N-terminus of the Fc domain by a hinge region or to the C-terminus of the Fc domain by a linking peptide or to the N-terminus of the heavy chain variable region of the binding domain that binds to TIGIT by a linking peptide.

In some embodiments, the bispecific antibody of the present disclosure includes:
a first polypeptide chain having at least 90%, 91%, 92%, 93%, 94%, 95%, 96%, 97%, 98%, 99% or 100% sequence identity to the amino acid sequence set forth in SEQ ID NO: 17, a second polypeptide chain having at least 90%, 91%, 92%, 93%, 94%, 95%, 96%, 97%, 98%, 99% or 100% sequence identity to the amino acid sequence set forth in SEQ ID NO: 18, and a third polypeptide chain having at least 90%, 91%, 92%, 93%, 94%, 95%, 96%, 97%, 98%, 99% or 100% sequence identity to the amino acid sequence set forth in SEQ ID NO: 19; or
a first polypeptide chain having at least 90%, 91%, 92%, 93%, 94%, 95%, 96%, 97%, 98%, 99% or 100% sequence identity to the amino acid sequence set forth in SEQ ID NO: 20, a second polypeptide chain having at least 90%, 91%, 92%, 93%, 94%, 95%, 96%, 97%, 98%, 99% or 100% sequence identity to the amino acid sequence set forth in SEQ ID NO: 18, and a third polypeptide chain having at least 90%, 91%, 92%, 93%, 94%, 95%, 96%, 97%, 98%, 99% or 100% sequence identity to the amino acid sequence set forth in SEQ ID NO: 21; or
a first polypeptide chain having at least 90%, 91%, 92%, 93%, 94%, 95%, 96%, 97%, 98%, 99% or 100% sequence identity to the amino acid sequence set forth in SEQ ID NO: 22, and a second polypeptide chain having at least 90%, 91%, 92%, 93%, 94%, 95%, 96%, 97%, 98%, 99% or 100% sequence identity to the amino acid sequence set forth in SEQ ID NO: 23; or
a first polypeptide chain having at least 90%, 91%, 92%, 93%, 94%, 95%, 96%, 97%, 98%, 99% or 100% sequence identity to the amino acid sequence set forth in SEQ ID NO: 24, and a second polypeptide chain having at least 90%, 91%, 92%, 93%, 94%, 95%, 96%, 97%, 98%, 99% or 100% sequence identity to the amino acid sequence set forth in SEQ ID NO: 25; or
two heavy chains having at least 90%, 91%, 92%, 93%, 94%, 95%, 96%, 97%, 98%, 99% or 100% sequence identity to the amino acid sequence set forth in SEQ ID NO: 26, and two light chains having at least 90%, 91%, 92%, 93%, 94%, 95%, 96%, 97%, 98%, 99% or 100% sequence identity to the amino acid sequence set forth in SEQ ID NO: 18; or
two heavy chains having at least 90%, 91%, 92%, 93%, 94%, 95%, 96%, 97%, 98%, 99% or 100% sequence identity to the amino acid sequence set forth in SEQ ID NO: 27, and two light chains having at least 90%, 91%, 92%, 93%, 94%, 95%, 96%, 97%, 98%, 99% or 100% sequence identity to the amino acid sequence set forth in SEQ ID NO: 18; or
two heavy chains having at least 90%, 91%, 92%, 93%, 94%, 95%, 96%, 97%, 98%, 99% or 100% sequence identity to the amino acid sequence set forth in SEQ ID NO: 28, and two light chains having at least 90%, 91%, 92%, 93%, 94%, 95%, 96%, 97%, 98%, 99% or 100% sequence identity to the amino acid sequence set forth in SEQ ID NO: 18; or
two heavy chains having at least 90%, 91%, 92%, 93%, 94%, 95%, 96%, 97%, 98%, 99% or 100% sequence identity to the amino acid sequence set forth in SEQ ID NO: 29, and two light chains having at least 90%, 91%, 92%, 93%, 94%, 95%, 96%, 97%, 98%, 99% or 100% sequence identity to the amino acid sequence set forth in SEQ ID NO: 18.

In yet another embodiment, the present disclosure provides a polynucleotide molecule whose nucleotide sequence is selected from:
(1) a nucleotide sequence encoding any one of the bispecific antibodies herein; and
(2) a complementary sequence of the nucleotide sequence of (1).

In yet another embodiment, the present disclosure provides an expression vector including the polynucleotide molecule described herein; In one embodiment, the expression vector is a eukaryotic expression vector.

In yet another embodiment, the present disclosure provides a host cell including the polynucleotide molecule described herein, or including the expression vector described herein, or expressing any one of the bispecific antibodies herein; In one embodiment, the host cell is a eukaryotic cell; In one embodiment, the host cell is a mammalian cell.

In yet another embodiment, the present disclosure provides a method for preparing any one of the bispecific antibodies herein, the method including culturing the host cell described herein under conditions suitable for the expression of any one of the bispecific antibodies herein to allow the host cell to express the bispecific antibody and collecting the expressed bispecific antibody from the host cell.

In yet another embodiment, the present disclosure provides a pharmaceutical composition including the bispecific antibody, polynucleotide molecule, expression vector or host cell according to any one of the embodiments herein, and a pharmaceutically acceptable carrier or excipient.

In yet another embodiment, the present disclosure provides use of the bispecific antibody, polynucleotide molecule, expression vector, host cell or pharmaceutical composition according to any one of the embodiments herein in the manufacture of a medicament for preventing or treating cancer.

In yet another aspect, the present disclosure provides the bispecific antibody, polynucleotide molecule, expression vector, host cell or pharmaceutical composition according to any one of the embodiments herein for use in the prevention or treatment of cancer.

In yet another embodiment, the present disclosure provides a method for preventing or treating cancer including administering to a subject in need thereof the bispecific antibody, polynucleotide molecule, expression vector, host cell or pharmaceutical composition according to any one of the embodiments herein.

In some embodiments, the cancer described in the present disclosure is associated with CD112R and/or TIGIT; In one embodiment, the cancer is selected from melanoma, renal cancer, prostate cancer, breast cancer, colon cancer, lung cancer, bone cancer, pancreatic cancer, skin cancer, head and neck cancer, uterine cancer, ovarian cancer and rectal cancer.

In yet another embodiment, the present disclosure provides a pharmaceutical combination including the bispecific antibody, polynucleotide molecule, expression vector, host cell or pharmaceutical composition according to any one of the embodiments herein, and one or more additional therapeutic agents.

In yet another embodiment, the present disclosure provides a kit including the bispecific antibody, polynucleotide molecule, expression vector, host cell or pharmaceutical composition according to any one of the embodiments herein.

### BRIEF DESCRIPTION OF THE DRAWINGS

FIG. 1: a schematic of the structure of a bispecific antibody molecule; 1a: a schematic of the structure of bispecific antibody molecule JS-1; 1b: a schematic of the structure of bispecific antibody molecule JS-3; 1c: a schematic of the structure of bispecific antibody molecule JS-5; 1d: a schematic of the structure of bispecific antibody molecule JS-9.

FIG. 2: the results of assaying bispecific antibodies for binding to human TIGIT (ELISA); 2a: the assay results for JS-1 (IgG4), JS-3 (IgG4) and JS-5 (IgG4); 2b: the assay result for JS-9 (IgG4).

FIG. 3: the results of assaying bispecific antibodies for binding to human CD112R (ELISA); 3a: the assay results for JS-1 (IgG4), JS-3 (IgG4) and JS-5 (IgG4); 3b: the assay result for JS-9 (IgG4).

FIG. 4: the results of assaying bispecific antibodies for activity for blocking the TIGIT/PVR interaction.

FIG. 5: the results of assaying bispecific antibodies for activity for blocking the CD112R/CD112 interaction.

FIG. 6: the results of assaying bispecific antibodies for activity in the CD112/TIGIT, CD155/TIGIT and CD112/CD112R luciferase reporter gene systems; 6a: the assay results for JS-1 (IgG4), JS-3 (IgG4) and JS-5 (IgG1); 6b: the assay result for JS-9 (IgG1).

FIG. 7: the tumor-inhibiting effects of bispecific antibodies in the mouse model of liver cancer built by transplanting H22-hPDL1 subcutaneously to BALB/c-hPD-1/hPDL1/hTIGIT/hPVRIG humanized mice.

### DETAILED DESCRIPTION

### Definitions

Unless otherwise stated, embodiments of the present disclosure will employ conventional techniques of molecular biology (including recombinant techniques), microbiology, cytobiology, biochemistry, and immunology.

In order to facilitate the understanding of the present disclosure, some technical and scientific terms are specifically defined as follows. Unless otherwise specifically defined herein, all technical and scientific terms used herein have the same meaning as commonly understood. For definitions and terminology in the art, specifically refer to Current Protocolsin Molecular Biology (Ausubel). Abbreviations for amino acid residues are standard three-letter and/or single-letter codes used in the art to denote one of the 20 commonly used L-amino acids. The singular forms used herein (including claims) include their plural forms, unless otherwise specified in the context explicitly.

The term “about” used in combination with a numerical value is intended to encompass the numerical values in a range from a lower limit less than the specified numerical value by 5% to an upper limit greater than the specified numerical value by 5%.

The term “and/or” should be understood as any one of the options or a combination of any two or more of the options.

The term “antigen” refers to a molecule or a portion of a molecule capable of being bound by the antibody of the present disclosure. An antigen may have one or more than one epitope.

The terms “CD112R”, “PVR-associated immunoglobulin domain”, “CD112 receptor”, “poliovirus receptor-associated immunoglobulin domain”, “Nectin-2 receptor”, “C7orf15”, and “transmembrane protein PVRIG” are all used interchangeably and include variants, subtypes, species homologs of human CD112R or CD112R of other species, and analogs having at least one epitope in common with CD112R, unless otherwise stated. The term includes unprocessed full-length CD112R and CD112R in any form resulting from intracellular processing. The term encompasses “full-length” unprocessed CD112R and CD112R in any form resulting from intracellular processing or any fragment thereof, such as a splice variant or an allelic variant.

In one embodiment, CD112R refers to full-length CD112R from humans or cynomolgus monkeys, or fragments thereof (such as mature fragments thereof lacking a signal peptide).

The term “human CD112R” refers to the human sequence CD112R, such as the complete amino acid sequence of the human CD112R under NCBI accession No. NM_024070. A human CD112R sequence may differ from the human CD112R under NCBI accession No. NM_024070 by having a conservative mutation or a mutation in a non-conservative region, and the CD112R has substantially the same biological function as the human CD112R under NCBI accession No. NM_024070.

The term “TIGIT”, short for T cell immunoreceptor with Ig and ITIM domains, refers to any natural TIGIT from any vertebrate animal (including mammals, such as primates (e.g., humans) and rodents (e.g., mice and rats)), unless otherwise indicated. This term encompasses “full-length” unprocessed TIGIT and TIGIT in any form resulting from intracellular processing or any fragment thereof. The term also includes variants of naturally occurring TIGIT, e.g., splice variants or allelic variants. In one embodiment, TIGIT refers to full-length TIGIT from humans and cynomolgus monkeys or fragments thereof (such as mature fragments thereof lacking a signal peptide). In one embodiment, human TIGIT refers to a mature TIGIT identical to a sequence of amino acid residues 22-244 (Genbank accession No. NP_776160.2) (amino acid residues 1-21 are leader peptides). In one embodiment, human TIGIT refers to a TIGIT extracellular domain identical to a sequence of amino acid residues 22-141 (Genbank accession No. NP_776160.2). In one embodiment, the cynomolgus monkey (Wacaca fascicuiaris) TIGIT refers to a mature TIGIT identical to a sequence of amino acid residues 22-245 (Genbank accession No. XP_005548158.1).

The term “percent (%) amino acid sequence identity”, or simply “identity”, is defined as the percentage of amino acid residues in a candidate amino acid sequence that are identical to those in a reference amino acid sequence after aligning the amino acid sequences (with gaps introduced if necessary) to achieve maximum percent sequence identity without considering any conservative substitution as part of sequence identity. Various methods in the art can be employed to perform sequence alignment to determine the percent amino acid sequence identity, for example, using computer software available to the public, such as BLAST, BLAST-2, ALIGN, or MEGALIGN (DNASTAR) software. Suitable parameters for measuring alignment may be determined, including any algorithm required to obtain maximum alignment for the full length of the aligned sequences.

The term “immune response” refers to the action of, for example, lymphocytes, antigen-presenting cells, phagocytes, granulocytes, and soluble macromolecules produced by the above cells or liver (including antibodies, cytokines and complements) that result in selective damage to, destruction of, or elimination from the human body of invading pathogens, cells or tissues infected with pathogens, cancerous cells, or, in the cases of autoimmunity or pathological inflammation, normal human cells or tissues.

The term “signal transduction pathway” or “signal transduction activity” refers to a biochemical causal relationship generally initiated by an interaction between proteins (such as binding of a growth factor to a receptor) and resulting in the transmission of a signal from one portion of a cell to another portion of the cell. In general, the transmission includes specific phosphorylation of one or more tyrosine, serine, or threonine residues on one or more proteins in a series of reactions causing signal transduction. The penultimate process typically involves a nuclear event, resulting in a change in gene expression.

The term “activity” or “bioactivity”, or the term “biological property” or “biological characteristic” can be used interchangeably herein and includes, but is not limited to, epitope/antigen affinity and specificity, the ability to neutralize or antagonize the activity of related antigens in vivo or in vitro, IC50, the in vivo stability of the antibody, and the immunogenic properties of the antibody. Other identifiable biological properties or characteristics of the antibody known in the art include, for example, cross-reactivity (i.e., cross-reactivity with non-human homologs of the targeted peptide, or with other proteins or tissues in general), and the ability to maintain a high expression level of the protein in mammalian cells. The aforementioned properties or characteristics are observed, determined, or assessed using techniques well known in the art, including but not limited to ELISA, FACS, or BIACORE plasma resonance analysis, unlimited in vitro or in vivo neutralization assays, receptor binding, cytokine or growth factor production and/or secretion, signal transduction, and immunohistochemistry of tissue sections of different sources (including human, primate or any other source).

The term “antibody” refers to any form of an antibody with the desired bioactivity. Thus, it is used in the broadest sense and specifically includes, but is not limited to, monoclonal antibodies (including full-length monoclonal antibodies), polyclonal antibodies, multispecific antibodies (e.g., bispecific antibodies), humanized antibodies, fully human antibodies, chimeric antibodies, and camelized single-domain antibodies. The basic antibody structural unit is known to include a tetramer. Each tetramer includes two identical pairs of polypeptide chains, each pair having a “light” chain (about 25 kDa) and a “heavy” chain (about 50-70 kDa). The amino-terminal portion or fragment of each chain may include a variable region of about 100-110 or more amino acids primarily responsible for antigen recognition. The carboxy-terminal portion or fragment of each chain may define a constant region primarily responsible for effector function. Human light chains are generally classified as κ and λ light chains. In addition, human heavy chains are generally classified as μ, δ, γ, α or ε chains, and isotypes of the antibody are defined as IgM, IgD, IgG, IgA and IgE, respectively. Within the light and heavy chains, the variable and constant regions are connected by a “J” region of about 12 or more amino acids, and the heavy chain also includes a “D” region of about 10 more amino acids. See generally chapter 7 of Fundamental Immunology (Ed. Paul, W., 2nd edition, Raven Press, N.Y. (1989)).

The term “isolated antibody” refers to the purified state of a binding compound, and, in this case, means that the molecule is substantially free of other biomolecules, such as nucleic acids, proteins, lipids, sugars, or other substances such as cell debris and growth medium. The term “isolate(d)” does not mean the complete absence of such substances or the absence of water, buffers, or salts, unless they are present in amounts that will significantly interfere with the experimental or therapeutic use of the binding compounds described herein.

The term “multispecific antibody” refers to an antibody including two or more antigen-binding domains and capable of binding to two or more different epitopes (e.g., two, three, four or more different epitopes) which may be on the same antigen or different antigens. Examples of multispecific antibodies include “bispecific antibodies” that bind to two different antigens or two different epitopes.

The term “full-length antibody” refers to an immunoglobulin molecule including four peptide chains when occurring naturally: two heavy (H) chains (about 50-70 kDa in full length) and two light (L) chains (about 25 kDa in full length) linked to each other by disulfide bonds. Each heavy chain consists of a heavy chain variable region (abbreviated herein as VH) and a heavy chain constant region (abbreviated herein as CH). The heavy chain constant region consists of 3 domains, CH1, CH2, and CH3. Each light chain consists of a light chain variable region (abbreviated herein as VL) and a light chain constant region. The light chain constant region consists of one domain CL. The VH and VL regions can be further divided into complementarity determining regions (CDRs) with high variability and more conservative regions called framework regions (FRs) that are spaced apart by the CDRs. Each VH or VL region consists of 3 CDRs and 4 FRs arranged in the following order from the amino terminus to the carboxyl terminus: FR1, CDR1, FR2, CDR2, FR3, CDR3, and FR4. The variable regions of the heavy and light chains contain binding domains that interact with antigens. The constant region of the antibody can mediate the binding of immunoglobulins to host tissues or factors, including the binding of various cells of the immune system (e.g., effector cells) to the first component (Clq) of classical complement system.

The terms “heavy chain constant region” and “CH” are used interchangeably herein and refer to a region including at least three heavy chain constant domains (CH1, CH2 and CH3). Non-limiting exemplary human heavy chain constant regions include γ, δ and α. Non-limiting exemplary human heavy chain constant regions further include ε and μ. Each heavy chain constant region corresponds to an antibody isotype. For example, an antibody including a γ constant region is an IgG antibody, an antibody including a δ constant region is an IgD antibody, and an antibody including an α constant region is an IgA antibody. In addition, an antibody including a μ constant region is an IgM antibody, and an antibody including an ε constant region is an IgE antibody. Certain isotypes can be further divided into subclasses. For example, IgG antibodies include, but are not limited to, IgG1 (including a γ1 constant region), IgG2 (including a γ2 constant region), IgG3 (including a γ3 constant region) and IgG4 (including a γ4 constant region) antibodies; IgA antibodies include, but are not limited to, IgA1 (including an al constant region) and IgA2 (including an α2 constant region) antibodies; and IgM antibodies include, but are not limited to, IgM1 and IgM2.

The terms “CH1 domain”, “CH1” and “heavy chain constant region 1” are used interchangeably herein and include the first (most amino termini) constant region domain of an immunoglobulin heavy chain that extends, e.g., from about positions 114-223 in the Kabat numbering system (EU positions 118-215). The CH1 domain is adjacent to the VH domain and the amino terminus of the hinge region of an immunoglobulin heavy chain molecule, and does not form a part of the Fc region of the immunoglobulin heavy chain, e.g., the human IgG1 CH1 domain, IgG2 CH1 domain, IgG3 CH1 domain and IgG4 CH1 domain; and naturally occurring variants thereof.

The terms “CH2 domain”, “CH2” and “heavy chain constant region 2” are used interchangeably herein and include the portion of a heavy chain immunoglobulin molecule that extends, e.g., from about positions 244-360 in the Kabat numbering system (EU positions 231-340). The CH2 domain is unique in that it is not closely paired with another domain. Rather, two N-linked branched carbohydrate chains are interposed between the two CH2 domains of an intact natural IgG molecule.

The terms “CH3 domain”, “CH3” and “heavy chain constant region 3” are used interchangeably herein and include the portion of a heavy chain immunoglobulin molecule that extends about 110 residues from a terminus of the CH2 domain, e.g., from about positions 361-476 of the Kabat numbering system (EU positions 341-445). The CH3 domain generally forms the C-terminal portion of the antibody. In some immunoglobulins, however, additional domains may extend from the CH3 domain to form the C-terminal portion of the molecule (e.g. the CH4 domain in the μ chain of IgM and the e chain of IgE).

The terms “light chain constant region” and “CL” are used interchangeably herein and refer to a region including a light chain constant domain (CL). Non-limiting exemplary light chain constant regions include λ and κ.

The term “antigen-binding fragment” of an antibody (“parent antibody”) includes a fragment or a derivative of the antibody, generally including at least one fragment of an antigen-binding region or variable region (e.g., one or more CDRs) of a parent antibody, which retains at least some of the binding specificity of the parent antibody. Examples of antigen-binding fragments include, but are not limited to, ScFab, Fab, Fab′, F(ab′)2, Fv and dsFv fragments; a diabody; a linear antibody; a single-chain antibody molecule, such as scFv; a nanobody (sdAb) and multispecific antibody formed by antibody fragments. The binding fragment or the derivative generally retains at least 10% of the antigen-binding activity of the parent antibody when the antigen-binding activity is expressed on a molar concentration basis. In one embodiment, the binding fragment or the derivative retains at least 20%, 50%, 70%, 80%, 90%, 95% or 100%, or more of the antigen-binding affinity of the parent antibody. It is also contemplated that the antigen-binding fragment of the antibody may include conservative or non-conservative amino acid substitutions that do not significantly alter its bioactivity (referred to as “conservative variants” or “function-conservative variants” of the antibody). The term “binding compound” refers to both the antibody and the binding fragment thereof. In the present application, the term “Fab” or “Fab fragment” generally refers to a fragment including a heavy chain variable domain (VH) and a light chain variable domain (VL) and further including the constant domain of the light chain (CL) and the first constant domain of the heavy chain (CH1). The term “Fab′” generally refers to a fragment that differs from Fab by having a few residues (including one or more cysteines from the antibody hinge region) at the carboxy terminus of the heavy chain CH1 domain. The term “F(ab)2” generally refers to a dimer of Fab′, an antibody fragment including two Fab fragments linked by a disulfide bridge in the hinge region. The term “Fv” or “Fv fragment” generally refers to the smallest antibody fragment that contains intact antigen recognition and binding sites; in some cases, the fragment may consist of a dimer of one heavy chain variable region and one light chain variable region in tight, non-covalent association. The term “dsFv” generally refers to a disulfide-stabilized Fv fragment whose bond between a single light chain variable region and a single heavy chain variable region is a disulfide bond. The term “sdAb fragment” generally refers to an antibody fragment consisting of a VH domain. The term “single-chain Fv” or “seFv” antibody refers to an antibody fragment including the VH and VL domains of an antibody, where these domains are present in a single polypeptide chain, and generally further include the polypeptide linker between the VH and VL domains, which enables the scFv to form the desired structure for antigen binding. The term “scFab fragment” or “ScFab” refers to a polypeptide consisting of an antibody heavy chain variable domain (VH), an antibody constant domain 1 (CH1), an antibody light chain variable domain (VL), an antibody light chain constant domain (CL) and a linker, and the antibody domains and the linker have one of the following sequences in the N-terminus-to-C-terminus direction: a) VH-CH1-linker-VL-CL, b) VL-CL-linker-VH-CH1, c) VH-CL-linker-VL-CH1, or d) VL-CH1-linker-VH-CL; and and the linker is a polypeptide of at least 30 amino acids, preferably 32 to 50 amino acids.

The terms “Fc”, “Fc region”, “Fc fragment” and “Fc domain” are used interchangeably herein to define a C-terminal region of an immunoglobulin heavy chain that contains at least a portion of a constant region. The term includes a natural sequence Fc region and a variant Fc region. A natural immunoglobulin “Fc domain” includes two or three constant domains, namely the CH2 domain, the CH3 domain and the optional CH4 domain. For example, in natural antibodies, the immunoglobulin Fc domain includes the second and third constant domains (the CH2 and CH3 domains) derived from the two heavy chains of IgG, IgA and IgD antibodies, or the second, third and fourth constant domains (the CH2, CH3 and CH4 domains) derived from the two heavy chains of IgM and IgE antibodies. The Fc region herein may also include a hinge region.

The term “hinge region” refers to the proline-rich, flexible polypeptide chain between CH1 and CH2 in an antibody.

The term “valency” refers to the number of potential antigen-binding sites in a polypeptide. Each antigen-binding site specifically binds to one antigenic molecule or specific site. When a polypeptide includes more than one antigen-binding site, each antigen-binding site may specifically bind to the same or different molecules (e.g., may bind to different ligands or different antigens, or different epitopes on the same antigen). The term “bivalent” means that the antibody includes two antigen-binding sites. The term “tetravalent” means that the antibody includes four antigen-binding sites.

The term “binding domain” or “antigen-binding domain” or “antigen-binding site” refers to a portion of an antibody that includes a region that specifically binds to, and is complementary to, part or the whole of an antigen. When the antigen is very large, the antibody may only be able to bind to a particular portion of the antigen, which is referred to as an epitope. A binding domain may include heavy and light chain variable domains (VH and VL), each including four conserved framework regions (FRs) and three CDRs. The CDRs vary in sequence and determine specificity for a particular antigen.

“Isotypes” of antibodies refer to types of antibodies (e.g., IgM, IgE and IgG (such as IgG1, IgG2 or IgG4)) provided by heavy chain constant region genes. Isotype also includes modified forms of one of these types in which modifications have been made to alter Fc function, for example, to enhance or attenuate effector function or binding to Fc receptors.

The term “epitope” refers to the region of an antigen to which an antibody binds. Epitopes can be formed from contiguous amino acids or noncontiguous amino acids juxtaposed by tertiary folding of a protein.

The terms “specific binding”, “selective binding”, “selectively bind to” and “specifically bind to” refer to the binding of an antibody to an epitope on a predetermined antigen. Specific binding can be measured, for example, by determining binding of a molecule compared to binding of a control molecule. The control molecule is generally a molecule having a similar structure without binding activity. For example, specific binding can be determined by competition with a control molecule that is similar to the target.

“Affinity” or “binding affinity” refers to inherent binding affinity that reflects the interaction between members of a binding pair. The affinity of molecule X for its partner Y can be generally represented by the equilibrium dissociation constant (KD), which is a ratio of the dissociation rate constant to the association rate constant (kdis and kon, respectively). Affinity can be measured using common methods known in the art. One specific method for measuring affinity is the ForteBio kinetic binding assay herein.

The term “not bind to” a protein or cell means not binding to the protein or cell, or not binding to it with high affinity, that is, binding to the protein or cell with a KD of 1.0×10⁻⁶ M or higher, more preferably 1.0×10⁻⁵ M or higher, more preferably 1.0×10⁻⁴ M or higher, 1.0×10⁻³ M or higher, and more preferably 1.0×10⁻² M or higher.

The term “high affinity” of IgG antibodies refers to a KD for the antigen of 1.0×10⁻⁶ M or less, preferably 5.0×10⁻⁸ M or less, more preferably 1.0×10⁻⁸ M or less, 5.0×10⁻⁹ M or less, and more preferably 1.0×10⁻⁹ M or less. For other antibody subtypes, “high affinity” binding may vary. For example, “high affinity” binding of the IgM subtype refers to a KD of 10⁻⁶ M or less, preferably 10⁻⁷ M or less, and more preferably 10⁻⁸ M or less.

The term “antibody-dependent cytotoxicity”, “antibody-dependent cell-mediated cytotoxicity” or “ADCC” refers to a cell-mediated immune defense in which the effector cells of the immune system actively lyse target cells, such as cancer cells, whose cell membrane surface antigens bind to antibodies, such as a Claudin18.2 antibody.

The term “complement-dependent cytotoxicity” or “CDC” refers to the effector function of IgG and IgM antibodies, which, when binding to surface antigens, trigger typical complement pathways, including formation of membrane attack complexes and lysis of target cells.

The term “nucleic acid” or “polynucleotide” refers to deoxyribonucleic acid (DNA) or ribonucleic acid (RNA) and polymers thereof in either single- or double-stranded form. Unless explicitly limited, the term includes nucleic acids containing known analogs of natural nucleotides that have binding properties similar to that of the reference nucleic acid and are metabolized in a manner similar to naturally occurring nucleotides (see U.S. Pat. No. 8,278,036 to Kariko et al., which discloses an mRNA molecule with uridine replaced by pseudouridine, a method for synthesizing the mRNA molecule, and a method for delivering a therapeutic protein in vivo). Unless otherwise specified, a particular nucleic acid sequence also implicitly includes conservatively modified variants thereof (e.g., degenerate codon substitutions), alleles, orthologs, SNPs, and complementary sequences as well as the sequence explicitly indicated. Specifically, degenerate codon substitutions can be achieved by generating sequences in which the third position of one or more selected (or all) codons is substituted with mixed bases and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “construct” refers to any recombinant polynucleotide molecule (such as plasmid, cosmid, virus, autonomously replicating polynucleotide molecule, phage, or linear or circular single- or double-stranded DNA or RNA polynucleotide molecule) derived from any source, capable of genomic integration or autonomous replication, and including a polynucleotide molecule where one or more polynucleotide molecules have been linked in a functionally operative manner (i.e., operably linked). The recombinant construct typically includes a polynucleotide of the present disclosure operably linked to transcription initiation regulatory sequences that will direct transcription of the polynucleotide in a host cell. Both heterologous and non-heterologous (i.e., endogenous) promoters can be used to direct expression of the nucleic acids of the present disclosure.

The term “vector” refers to any recombinant polynucleotide construct that can be used for transformation purposes (i.e., the introduction of heterologous DNA into a host cell). One type of vector is a “plasmid”, which refers to a circular double-stranded DNA loop into which additional DNA segments can be ligated. Another type of vector is a viral vector, in which additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into genome of the host cell upon introduction into a host cell, and are to replicate along with the host genome. In addition, certain vectors are capable of directing the expression of operably linked genes. Such vectors are referred to herein as “expression vectors”.

The term “expression vector” refers to a nucleic acid molecule capable of replicating and expressing a target gene when transformed, transfected or transduced into a host cell. The expression vector includes one or more phenotypic selectable markers and an origin of replication to ensure maintenance of the vector and to provide amplification in the host if needed.

Unless otherwise or explicitly specified in the context, “activation”, “stimulation” and “treatment” for a cell or a receptor may have the same meaning. For example, the cell or the receptor is activated, stimulated or treated with a ligand. “Ligands” include natural and synthetic ligands, such as cytokines, cytokine variants, analogs, mutant proteins, and binding compounds derived from antibodies. “Ligands” also include small molecules, such as peptidomimetics of cytokines and peptidomimetics of antibodies. “Activation” may refer to the activation of a cell regulated by internal mechanisms and external or environmental factors. “Response/reaction”, e.g., a response of a cell, a tissue, an organ or an organism, includes changes in biochemical or physiological behaviors (e.g., concentration, density, adhesion or migration, gene expression rate, or differentiation state within a biological compartment), where the changes are associated with activation, stimulation or treatment, or are associated with an internal mechanism such as genetic programming.

As used herein, the term “treat”, “treating” or “treatment” of any disease or disorder refers in one embodiment to ameliorating the disease or disorder (i.e., slowing or arresting or reducing the progression of the disease or at least one of its clinical symptoms). In another embodiment, “treat”, “treating” or “treatment” refers to ameliorating or alleviating at least one physical parameter, including those physical parameters that may not be discernible by the patient. In another embodiment, “treat”, “treating” or “treatment” refers to modulating the disease or disorder, physically (e.g., stabilization of discernible symptoms), physiologically (e.g., stabilization of physical parameters), or both. Unless explicitly described herein, methods for assessing treatment and/or prevention of disease are generally known in the art.

“Subject” includes any human or non-human animal. The term “non-human animal” includes all vertebrates, e.g., mammals and non-mammals, such as non-human primates, sheep, dog, cat, horse, cattle, chicken, amphibians, and reptiles. As used herein, the term “cyno” refers to a cynomolgus monkey.

Administration “in combination with” one or more other therapeutic agents includes simultaneous (co-) administration and sequential administration in any order.

“Therapeutically effective amount”, “therapeutically effective dose” and “effective amount” refer to an amount of the bispecific antibody of the present disclosure that is effective in preventing or ameliorating one or more symptoms of a disease or condition or the progression of the disease or condition when administered alone or in combination with other therapeutic drugs to a cell, a tissue or a subject. The therapeutically effective dose also refers to an amount of the bispecific antibody sufficient to cause an improvement in symptoms, e.g., an amount for treating, curing, preventing or improving a related condition or promoting the treatment, cure, prevention or improvement of such condition. When an active ingredient is administered to an individual alone, a therapeutically effective dose refers to the amount of the ingredient. In the case of administration in combination, a therapeutically effective dose refers to the combined amount of active ingredients that produces a therapeutic effect, regardless of whether these active ingredients are administered in combination, sequentially or simultaneously. An effective amount of a therapeutic agent will increase a diagnostic index or parameter by at least 10%, generally at least 20%, preferably at least about 30%, more preferably at least 40%, and most preferably at least 50%.

“Cancer” and “cancerous” refer to or describe the physiological condition in mammals which is typically characterized by unregulated cell growth. Included in this definition are benign and malignant cancers as well as dormant tumors or micrometastases. Cancer may be a solid tumor or a hematologic tumor, including but not limited to, carcinoma, lymphoma, blastoma, sarcoma and leukemia. More specific examples of such cancers include squamous cell carcinoma, lung cancer (including small cell lung cancer, non-small cell lung cancer, adenocarcinoma of the lung and squamous carcinoma of the lung), peritoneal cancer, hepatocellular cancer, cancer of the stomach or gastric cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial cancer or uterine cancer, salivary gland carcinoma, renal cancer or cancer of the kidney, prostatic cancer, vulval cancer, thyroid cancer, cancer of the liver, and various types of head and neck cancers, as well as B-cell lymphoma (including low-grade/follicular non-Hodgkin lymphoma (NHL), small lymphocytic (SL) NHL, intermediate-grade/follicular NHL, intermediate-grade diffuse NHL, high-grade immunoblastic NHL, high-grade lymphoblastic NHL, high-grade small non-cleaved cell NHL, bulky disease NHL, mantle cell lymphoma, AIDS-related lymphomas, and Waldenstrom macroglobulinemia), chronic lymphocytic leukemia (CLL), acute lymphoblastic leukemia (ALL), hairy cell leukemia, chronic myeloblastic leukemia, post-transplant lymphoproliferative disorder (PTLD), and abnormal vascular proliferation associated with phakomatoses, edema (such as associated with brain tumors) and Meigs syndrome.

“Cytokine storm” refers to the rapid generation of a variety of cytokines such as TNF-α, IL-1, IL-6, IL-12, IFN-α, IFN-β, IFN-γ, MCP-1 and IL-8 in large quantities in body fluids, which is a major cause of acute respiratory distress syndrome and multiple organ failure. Cytokine release syndrome (CRS) refers to a group of clinical syndromes caused by the activation and lysis of lymphocytes and release of cytokines in large quantities following treatment with monoclonal antibodies, cytokines, etc. or infection.

Antibody

The term “anti-CD112R antibody”, “anti-CD112R”, “CD112R antibody”, “binding to CD112R” or “CD112R-binding” refers to an antibody that is capable of binding to a CD112R protein or a fragment thereof with sufficient affinity such that the antibody can be used as a diagnostic and/or therapeutic agent targeting CD112R.

The term “anti-TIGIT antibody”, “anti-TIGIT”, “TIGIT antibody”, “binding to TIGIT” or “TIGIT-binding” refers to an antibody that is capable of binding to a TIGIT protein or a fragment thereof with sufficient affinity such that the antibody can be used as a diagnostic and/or therapeutic agent targeting TIGIT.

The CDR sequences or variable region sequence of the binding domain of the present disclosure that binds to CD112R includes the CDR sequences or variable region sequence of any one of the anti-CD112R antibodies described in Application No. PCT/CN2022/075502, the disclosure of which is incorporated herein by reference in its entirety. In some embodiments, the CDR sequences or variable region sequence of the binding domain of the bispecific antibody of the present disclosure that binds to CD112R includes the CDR sequences or variable region sequence of the antibody hu16, hu52 or hu59 described in PCT/CN2022/075502. More specifically, the amino acid sequences of hu16, hu52 and hu59 are as follows:

Hu16-LC: (the variable region sequence is indicated in bold type, and the
CDR sequences are underlined)
SEQ ID NO: 32
DIVMTQSPDSLAVSLGERATINCKASQSVSNDVAWYQQKPGQPPKLLIYYVSH
RFTGVPDRFSGSGSGTDFTLTISSLQAEDVAVYFCHQAYRSPWTFGQGTKLEIKRT
VAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC;
Hu16-HC-IgG4: (the variable region sequence is indicated in bold type,
and the CDR sequences are underlined)
SEQ ID NO: 33
EVQLVESGGGLVKPGGSLRLSCAASGFTFSSYHMSWVRQAPGKGLELVSAIS
RDGDSTYYPDTVKGRFTISRDNAKNSLYLQMNSLRAEDTAVYYCSRHEDYFGFAM
DSWGQGTLVTVSSASTKGPSVFPLAPCSRSTSESTAALGCLVKDYFPEPVTVSWNSGA
LTSGVHTFPAVLQSSGLYSLSSVVTVPSSSLGTKTYTCNVDHKPSNTKVDKRVESKYGP
PCPPCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK;
Hu16-HC-IgG1: (the variable region sequence is indicated in bold type,
and the CDR sequences are underlined)
SEQ ID NO: 34
EVQLVESGGGLVKPGGSLRLSCAASGFTFSSYHMSWVRQAPGKGLELVSAIS
RDGDSTYYPDTVKGRFTISRDNAKNSLYLQMNSLRAEDTAVYYCSRHEDYFGFAM
DSWGQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK;
Hu52-LC: (the variable region sequence is indicated in bold type, and the
CDR sequences are underlined)
SEQ ID NO: 35
DIVMTQSPDSLAVSLGERATINCKASQSVSNDVAWYQQKPGQPPKLLIYYVSH
RFTGVPDRFSGSGSGTDFTLTISSLQAEDVAVYFCHQAYRSPWTFGQGTKLEIKRT
VAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC;
Hu52-HC-IgG4: (the variable region sequence is indicated in bold type,
and the CDR sequences are underlined)
SEQ ID NO: 36
EVQLVESGGGLVKPGGSLRLSCAASGFTFSSYHMSWVRQAPGKGLELVSAIN
SNGINTYYLDTVKGRFTISRDNAKNSLYLQMNSLRAEDTAVYYCARHEDYYGFAM
DYWGQGTLVTVSSASTKGPSVFPLAPCSRSTSESTAALGCLVKDYFPEPVTVSWNSGA
LTSGVHTFPAVLQSSGLYSLSSVVTVPSSSLGTKTYTCNVDHKPSNTKVDKRVESKYGP
PCPPCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK;
Hu52-HC-IgG1: (the variable region sequence is indicated in bold type,
and the CDR sequences are underlined)
SEQ ID NO: 37
EVQLVESGGGLVKPGGSLRLSCAASGFTFSSYHMSWVRQAPGKGLELVSAIN
SNGINTYYLDTVKGRFTISRDNAKNSLYLQMNSLRAEDTAVYYCARHEDYYGFAM
DYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK;
Hu59-LC: (the variable region sequence is indicated in bold type, and the
CDR sequences are underlined)
SEQ ID NO: 38
DIQMTQSPSSLSASVGDRVTITCKASQSVSNDVAWYQQKPGKAPKLLIYYVSN
RFTGVPSRFSGSGSGTDFTLTISSLQPEDFATYFCQQAYRSPWTFGQGTKLEIKRTV
AAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC;
Hu59-HC-IgG4: (the variable region sequence is indicated in bold type,
and the CDR sequences are underlined)
SEQ ID NO: 39
EVQLVESGGGLVQPGGSLRLSCAASGFTFSSYHMSWIRQAPGKGLELVAAINS
NGINTYYLDTVKGRFTISRDNAKNSLYLQMNSLRAEDTAVYYCARHEDYYGFAM
DYWGQGTLVTVSSASTKGPSVFPLAPCSRSTSESTAALGCLVKDYFPEPVTVSWNSGA
LTSGVHTFPAVLQSSGLYSLSSVVTVPSSSLGTKTYTCNVDHKPSNTKVDKRVESKYGP
PCPPCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK;
Hu59-HC-IgG1: (the variable region sequence is indicated in bold type,
and the CDR sequences are underlined)
SEQ ID NO: 40
EVQLVESGGGLVQPGGSLRLSCAASGFTFSSYHMSWIRQAPGKGLELVAAINS
NGINTYYLDTVKGRFTISRDNAKNSLYLQMNSLRAEDTAVYYCARHEDYYGFAM
DYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK.

The CDR sequences or variable region sequence of the binding domain of the present disclosure that binds to TIGIT includes the CDR sequences or variable region sequence of any one of the anti-TIGIT antibodies described in Application No. PCT/CN2020/101883, the disclosure of which is incorporated herein by reference in its entirety. In some embodiments, the CDR sequences or variable region sequence of the binding domain of the bispecific antibody of the present disclosure that binds to TIGIT includes the CDR sequences or variable region sequence of the antibody hu20 or hu3 described in PCT/CN2020/101883.

In one embodiment, the present disclosure provides a bispecific antibody including a binding domain that binds to CD112R and a binding domain that binds to TIGIT, and the binding domain that binds to CD112R includes:
HCDR1, HCDR2 and HCDR3 of the amino acid sequence set forth in SEQ ID NO: 1, and/or LCDR1, LCDR2 and LCDR3 of the amino acid sequence set forth in SEQ ID NO: 2;
and the HCDR1, HCDR2, HCDR3, LCDR1, LCDR2 and LCDR3 are defined according to the Kabat, IMGT, Chothia, AbM or Contact numbering system.

The amino acid sequence of SEQ ID NO: 1 is:
EVQLVESGGGLVKPGGSLRLSCAASGFTFSSYHMSWVRQAPGKCLELVS
AINSNGINTYYLDTVKGRFTISRDNAKNSLYLQMNSLRAEDTAVYYCAR
HEDYYGFAMDYWGQGTLVTVSS;
the amino acid sequence of SEQ ID NO: 2 is:
DIVMTQSPDSLAVSLGERATINCKASQSVSNDVAWYQQKPGQPPKLLIY
YVSHRFTGVPDRFSGSGSGTDFTLTISSLQAEDVAVYFCHQAYRSPWTF
GCGTKLEIK.

In some embodiments, the binding domain of the present disclosure that binds to CD112R includes, according to the Kabat numbering system: an HCDR1 whose amino acid sequence is set forth in SEQ ID NO: 3, an HCDR2 whose amino acid sequence is set forth in SEQ ID NO: 4, an HCDR3 whose amino acid sequence is set forth in SEQ ID NO: 5, an LCDR1 whose amino acid sequence is set forth in SEQ ID NO: 6, an LCDR2 whose amino acid sequence is set forth in SEQ ID NO: 7, and an LCDR3 whose amino acid sequence is set forth in SEQ ID NO: 8.

In some embodiments, the amino acid sequences of SEQ ID NOs: 3-8 are as follows:

| | |
|---|---|
| SEQ ID NO: 3 | SYHMS |
| SEQ ID NO: 4 | AINSNGINTYYLDTVKG |
| SEQ ID NO: 5 | HEDYYGFAMDY |
| SEQ ID NO: 6 | KASQSVSNDVA |
| SEQ ID NO: 7 | YVSHRFT |
| SEQ ID NO: 8 | HQAYRSPWT |

In some embodiments, the binding domain of the present disclosure that binds to TIGIT includes:
HCDR1, HCDR2 and HCDR3 of the amino acid sequence set forth in SEQ ID NO: 9, and/or LCDR1, LCDR2 and LCDR3 of the amino acid sequence set forth in SEQ ID NO: 10;
and the HCDR1, HCDR2, HCDR3, LCDR1, LCDR2 and LCDR3 are defined according to the Kabat, IMGT, Chothia, AbM or Contact numbering system.

The amino acid sequence of SEQ ID NO: 9 is:
QVQLVQSGAEVKKPGASVKVSCKTSGYAFTEYTMHWVRQAPGKGLEWMG
GINPNTGGTTYNQKFQGRVTLTVDKSSSTAYMELSSLRSEDTVVYYCAK
LLRLMYYFDYWGQGTLVTVSS;
the amino acid sequence of SEQ ID NO: 10 is:
DIQMTQSPSSLSASVGDRVTITCQASQDVRTAVAWYQQKPGKAPKLLIY
SASYRYTGVPSRFSGSGSGTDFTFTISSLQPEDIATYYCHQHYITPWTF
GGGTKVEIK.

In some embodiments, the binding domain of the present disclosure that binds to TIGIT includes, according to the Kabat numbering system: an HCDR1 whose amino acid sequence is set forth in SEQ ID NO: 11, an HCDR2 whose amino acid sequence is set forth in SEQ ID NO: 12, an HCDR3 whose amino acid sequence is set forth in SEQ ID NO: 13, an LCDR1 whose amino acid sequence is set forth in SEQ ID NO: 14, an LCDR2 whose amino acid sequence is set forth in SEQ ID NO: 15, and an LCDR3 whose amino acid sequence is set forth in SEQ ID NO: 16.

In some embodiments, the amino acid sequences of SEO ID NOs: 11-16 are as follows:

| | |
|---|---|
| SEQ ID NO:11 | EYTMH |
| SEQ ID NO:12 | GINPNTGGTTYNQKFQG |
| SEQ ID NO:13 | LLRLMYYFDY |
| SEQ ID NO:14 | QASQDVRTAVA |
| SEQ ID NO:15 | SASYRYT |
| SEQ ID NO:16 | HQHYITPWT |

In some embodiments, the binding domain of the present disclosure that binds to CD112R includes:
a heavy chain variable region having at least 90%, 91%, 92%, 93%, 94%, 95%, 96%, 97%, 98%, 99% or 100% sequence identity to the amino acid sequence set forth in SEQ ID NO: 1, and a light chain variable region having at least 90%, 91%, 92%, 93%, 94%, 95%, 96%, 97%, 98%, 99% or 100% sequence identity to the amino acid sequence set forth in SEQ ID NO: 2; or
a heavy chain variable region having at least 90%, 91%, 92%, 93%, 94%, 95%, 96%, 97%, 98%, 99% or 100% sequence identity to the amino acid sequence set forth in SEQ ID NO: 30, and a light chain variable region having at least 90%, 91%, 92%, 93%, 94%, 95%, 96%, 97%, 98%, 99% or 100% sequence identity to the amino acid sequence set forth in SEQ ID NO: 31.

In some embodiments, the amino acid sequence of
SEQ ID NO: 30 is:
EVQLVESGGGLVKPGGSLRLSCAASGFTFSSYHMSWVRQAPGKGLELVS
AINSNGINTYYLDTVKGRFTISRDNAKNSLYLQMNSLRAEDTAVYYCAR
HEDYYGFAMDYWGQGTLVTVSS;
the amino acid sequence of SEQ ID NO: 31 is:
DIVMTQSPDSLAVSLGERATINCKASQSVSNDVAWYQQKPGQPPKLLIY
YVSHRFTGVPDRFSGSGSGTDFTLTISSLQAEDVAVYFCHQAYRSPWTF
GQGTKLEIK.

In some embodiments, the binding domain of the present disclosure that binds to CD112R includes:
a heavy chain variable region whose amino acid sequence is set forth in SEQ ID NO: 1, and a light chain variable region whose amino acid sequence is set forth in SEQ ID NO: 2; or
a heavy chain variable region whose amino acid sequence is set forth in SEQ ID NO: 30, and a light chain variable region whose amino acid sequence is set forth in SEQ ID NO: 31.

In some embodiments, the binding domain of the present disclosure that binds to TIGIT includes: a heavy chain variable region having at least 90%, 91%, 92%, 93%, 94%, 95%, 96%, 97%, 98%, 99% or 100% sequence identity to the amino acid sequence set forth in SEQ ID NO: 9, and a light chain variable region having at least 90%, 91%, 92%, 93%, 94%, 95%, 96%, 97%, 98%, 99% or 100% sequence identity to the amino acid sequence set forth in SEQ ID NO: 10.

In some embodiments, the binding domain of the present disclosure that binds to TIGIT includes: a heavy chain variable region whose amino acid sequence is set forth in SEQ ID NO: 9, and a light chain variable region whose amino acid sequence is set forth in SEQ ID NO: 10.

In some embodiments, the binding domain of the present disclosure that binds to CD112R is an ScFab fragment, a Fab fragment, an scFv fragment or an Fv fragment, and the binding domain that binds to TIGIT is an ScFab fragment, a Fab fragment, an scFv fragment or an Fv fragment.

In some embodiments, the binding domain of the present disclosure that binds to CD112R is an ScFab fragment or an scFv fragment, and the binding domain that binds to TIGIT is an ScFab fragment or a Fab fragment.

In some embodiments, the binding domain of the present disclosure that binds to CD112R is an ScFab fragment, and the binding domain that binds to TIGIT is an ScFab fragment; or the binding domain that binds to CD112R is an scFv fragment, and the binding domain that binds to TIGIT is a Fab fragment.

In some embodiments, the bispecific antibody of the present disclosure further includes an Fc domain.

In some embodiments, the Fc domain of the present disclosure is an IgG Fc domain, such as an IgG1 Fc domain, an IgG2 Fc domain, an IgG3 Fc domain or an IgG4 Fc domain.

In some embodiments, the bispecific antibody of the present disclosure is a bivalent or tetravalent bispecific antibody; In one embodiment, the binding domain that binds to TIGIT is linked to the N-terminus of the Fc domain by a hinge region, and the binding domain that binds to CD112R is linked to the N-terminus of the Fc domain by a hinge region or to the C-terminus of the Fc domain by a linking peptide or to the N-terminus of the heavy chain variable region of the binding domain that binds to TIGIT by a linking peptide.

In some embodiments, the bispecific antibody of the present disclosure has three polypeptide chains, and the first polypeptide chain has, from N-terminus to C-terminus, VH1-CH1-hinge region-first Fc region, the second polypeptide chain has, from N-terminus to C-terminus, VL1-CL, and the third polypeptide chain has, from N-terminus to C-terminus, VL2-linking peptide-VH2-linking peptide-hinge region-second Fc region. In one embodiment, the VH1-CH1 of the first polypeptide chain and the VL1-CL of the second polypeptide chain form the binding domain that binds to TIGIT, and the VL2-linking peptide-VH2 of the third polypeptide chain forms the binding domain that binds to CD112R.

In some embodiments, the bispecific antibody of the present disclosure has two polypeptide chains, and the first polypeptide chain has, from N-terminus to C-terminus, VL1-CL-linking peptide-VH1-CH1-hinge region-first Fc region, and the second polypeptide chain has, from N-terminus to C-terminus, VL2-CL-linking peptide-VH2-CH1-hinge region-second Fc region. In one embodiment, the VL1-CL-linking peptide-VH1-CH1 of the first polypeptide chain forms the binding domain that binds to TIGIT, and the VL2-CL-linking peptide-VH2-CH1 of the second polypeptide chain forms the binding domain that binds to CD112R.

In some embodiments, the bispecific antibody of the present disclosure has two identical heavy chains and two identical light chains, and the heavy chains have, from N-terminus to C-terminus, VH1-CH1-hinge region-Fc region-linking peptide-VL2-linking peptide-VH2 or VL2-linking peptide-VH2-linking peptide-VH1-CH1-hinge region-Fc region, and the light chains have, from N-terminus to C-terminus, VL1-CL. In one embodiment, the VH1-CH1 of the heavy chains and the VL1-CL of the light chains form the binding domain that binds to TIGIT, and the VL2-linking peptide-VH2 of the heavy chains forms the binding domain that binds to CD112R.

In some embodiments, the bispecific antibody of the present disclosure includes:
a first polypeptide chain having at least 90%, 91%, 92%, 93%, 94%, 95%, 96%, 97%, 98%, 99% or 100% sequence identity to the amino acid sequence set forth in SEQ ID NO: 17, a second polypeptide chain having at least 90%, 91%, 92%, 93%, 94%, 95%, 96%, 97%, 98%, 99% or 100% sequence identity to the amino acid sequence set forth in SEQ ID NO: 18, and a third polypeptide chain having at least 90%, 91%, 92%, 93%, 94%, 95%, 96%, 97%, 98%, 99% or 100% sequence identity to the amino acid sequence set forth in SEQ ID NO: 19; or
a first polypeptide chain having at least 90%, 91%, 92%, 93%, 94%, 95%, 96%, 97%, 98%, 99% or 100% sequence identity to the amino acid sequence set forth in SEQ ID NO: 20, a second polypeptide chain having at least 90%, 91%, 92%, 93%, 94%, 95%, 96%, 97%, 98%, 99% or 100% sequence identity to the amino acid sequence set forth in SEQ ID NO: 18, and a third polypeptide chain having at least 90%, 91%, 92%, 93%, 94%, 95%, 96%, 97%, 98%, 99% or 100% sequence identity to the amino acid sequence set forth in SEQ ID NO: 21; or
a first polypeptide chain having at least 90%, 91%, 92%, 93%, 94%, 95%, 96%, 97%, 98%, 99% or 100% sequence identity to the amino acid sequence set forth in SEQ ID NO: 22, and a second polypeptide chain having at least 90%, 91%, 92%, 93%, 94%, 95%, 96%, 97%, 98%, 99% or 100% sequence identity to the amino acid sequence set forth in SEQ ID NO: 23; or
a first polypeptide chain having at least 90%, 91%, 92%, 93%, 94%, 95%, 96%, 97%, 98%, 99% or 100% sequence identity to the amino acid sequence set forth in SEQ ID NO: 24, and a second polypeptide chain having at least 90%, 91%, 92%, 93%, 94%, 95%, 96%, 97%, 98%, 99% or 100% sequence identity to the amino acid sequence set forth in SEQ ID NO: 25; or
two heavy chains having at least 90%, 91%, 92%, 93%, 94%, 95%, 96%, 97%, 98%, 99% or 100% sequence identity to the amino acid sequence set forth in SEQ ID NO: 26, and two light chains having at least 90%, 91%, 92%, 93%, 94%, 95%, 96%, 97%, 98%, 99% or 100% sequence identity to the amino acid sequence set forth in SEQ ID NO: 18; or
two heavy chains having at least 90%, 91%, 92%, 93%, 94%, 95%, 96%, 97%, 98%, 99% or 100% sequence identity to the amino acid sequence set forth in SEQ ID NO: 27, and two light chains having at least 90%, 91%, 92%, 93%, 94%, 95%, 96%, 97%, 98%, 99% or 100% sequence identity to the amino acid sequence set forth in SEQ ID NO: 18; or
two heavy chains having at least 90%, 91%, 92%, 93%, 94%, 95%, 96%, 97%, 98%, 99% or 100% sequence identity to the amino acid sequence set forth in SEQ ID NO: 28, and two light chains having at least 90%, 91%, 92%, 93%, 94%, 95%, 96%, 97%, 98%, 99% or 100% sequence identity to the amino acid sequence set forth in SEQ ID NO: 18; or
two heavy chains having at least 90%, 91%, 92%, 93%, 94%, 95%, 96%, 97%, 98%, 99% or 100% sequence identity to the amino acid sequence set forth in SEQ ID NO: 29, and two light chains having at least 90%, 91%, 92%, 93%, 94%, 95%, 96%, 97%, 98%, 99% or 100% sequence identity to the amino acid sequence set forth in SEQ ID NO: 18.

In some embodiments, the bispecific antibody of the present disclosure includes:
a first polypeptide chain whose amino acid sequence is set forth in SEQ ID NO: 17, a second polypeptide chain whose amino acid sequence is set forth in SEQ ID NO: 18, and a third polypeptide chain whose amino acid sequence is set forth in SEQ ID NO: 19; or
a first polypeptide chain whose amino acid sequence is set forth in SEQ ID NO: 20, a second polypeptide chain whose amino acid sequence is set forth in SEQ ID NO: 18, and a third polypeptide chain whose amino acid sequence is set forth in SEQ ID NO: 21; or
a first polypeptide chain whose amino acid sequence is set forth in SEQ ID NO: 22, and a second polypeptide chain whose amino acid sequence is set forth in SEQ ID NO: 23; or
a first polypeptide chain whose amino acid sequence is set forth in SEQ ID NO: 24, and a second polypeptide chain whose amino acid sequence is set forth in SEQ ID NO: 25; or
two heavy chains whose amino acid sequences are set forth in SEQ ID NO: 26, and two light chains whose amino acid sequences are set forth in SEQ ID NO: 18; or
two heavy chains whose amino acid sequences are set forth in SEQ ID NO: 27, and two light chains whose amino acid sequences are set forth in SEQ ID NO: 18; or
two heavy chains whose amino acid sequences are set forth in SEQ ID NO: 28, and two light chains whose amino acid sequences are set forth in SEQ ID NO: 18; or
two heavy chains whose amino acid sequences are set forth in SEQ ID NO: 29, and two light chains whose amino acid sequences are set forth in SEQ ID NO: 18.

The precise amino acid sequence boundaries of the variable region CDRs of the antibodies of the present disclosure can be determined using any of many well-known schemes, including Chothia based on the three-dimensional structure of antibodies and the topology of the CDR loops (Chothia et al., (1989) Nature 342: 877-883; Al-Lazikani et al., Standard conformations for the canonical structures of immunoglobulins, Journal of Molecular Biology, 273, 927-948 (1997)), Kabat based on antibody sequence variability (Kabat et al., Sequences of Proteins of Immunological Interest, 4th edition, U.S. Department of Health and Human Services, National Institutes of Health (1987)), AbM (University of Bath), Contact (University College London), International ImMunoGeneTics database (IMGT) (1999 Nucleic Acids Research, 27, 209-212), and North CDR definition based on the affinity propagation clustering using a large number of crystal structures. The boundaries of the CDRs of the antibodies disclosed herein can be determined according to any scheme (e.g., different assignment systems or combinations) in the art.

It should be noted that the boundaries of the CDRs of the variable regions of the same antibody obtained based on different assignment systems may differ. That is, the CDR sequences of the variable regions of the same antibody defined under different assignment systems are different. Thus, when it comes to defining an antibody with a particular CDR sequence defined in the present disclosure, the scope of the antibody also encompasses antibodies whose variable region sequences include the particular CDR sequence but whose claimed CDR boundaries differ from the particular CDR boundaries defined in the present disclosure due to the application of different schemes (e.g., different assignment systems or combinations).

Antibodies with different specificities (i.e., different binding sites for different antigens) have different CDRs. However, although CDRs vary from antibody to antibody, only a limited number of amino acid positions within a CDR are directly involved in antigen binding. The smallest overlapping region can be determined using at least two of the Kabat, Chothia, AbM, Contact and North methods, to provide a “smallest binding unit” for antigen binding. The smallest binding unit may be a sub-portion of the CDR. As will be clear the residues in the remainder of the CDR sequences can be determined by the antibody structure and protein folding. Thus, variants of any of the CDRs presented herein are also contemplated by the present disclosure. For example, in a variant of one CDR, the amino acid residue of the smallest binding unit may remain unchanged, while the remaining CDR residues defined according to Kabat or Chothia may be replaced by conservative amino acid residues.

For the humanized antibodies of the present disclosure, murine CDR regions can be inserted into human germline framework regions using methods known in the art. See U.S. Pat. No. 5,225,539 to Winter et al., and U.S. Pat. Nos. 5,530,101; 5,585,089; 5,693,762 and 6,180,370 to Queen et al.

In some embodiments, the amino acid change includes an amino acid deletion, insertion, or substitution. In some embodiments, the bispecific antibody of the present disclosure includes those antibodies whose amino acid sequences have been mutated by amino acid deletion, insertion or substitution but still have at least about 90%, 91%, 92%, 93%, 94%, 95%, 96%, 97%, 98%, 99% or 100% identity to the antibodies described above (particularly in the CDR regions set forth in the above sequences). In some embodiments, when compared to the CDR regions set forth in a particular sequence, the antibodies of the present disclosure have no more than 1, 2, 3, 4, or 5 amino acid mutations (deletions, insertions or substitutions) in the CDR regions.

In some embodiments, polynucleotides encoding the antibodies disclosed herein include those that have been mutated by nucleotide deletion, insertion or substitution but still have at least about 60%, 70%, 80%, 90%, 95% or 100% identity to the corresponding CDR coding regions set forth in the above sequences.

In some embodiments, one or more amino acid modifications may be introduced into an Fc region of an antibody provided herein, thus producing an Fc region variant. The Fc region variant may include human Fc region sequences (e.g., human IgG1, IgG2, IgG3, or IgG4 Fc regions) which include amino acid modifications (e.g., substitutions) at one or more amino acid positions.

In some embodiments, antibodies modified by cysteine engineering may need to be produced, such as “sulfo-MAb”, and one or more residues of the antibodies are substituted by cysteine residues.

In some embodiments, the antibodies provided herein can be further modified to contain other non-protein moieties known in the art and readily available. Suitable moieties for antibody derivatization include, but are not limited to, water-soluble polymers. Non-limiting examples of water-soluble polymers include, but are not limited to, polyethylene glycol (PEG), ethylene glycol/propylene glycol copolymer, carboxymethyl cellulose, glucan, polyvinyl alcohol, polyvinylpyrrolidone, poly-1,3-dioxane, poly-1,3,6-trioxane, ethylene/maleic anhydride copolymer, polyamino acid (homopolymer or random copolymer), and glucan or poly(n-vinylpyrrolidone)polyethylene glycol, propylene glycol homopolymer, polypropylene oxide/ethylene oxide copolymer, polyoxyethylated polyol (such as glycerol), polyvinyl alcohol, and mixtures thereof.

The Fc region of the antibody of the present disclosure may be a human Fc region. The Fc region of the bispecific antibody of the present disclosure may be of any isotype, including but not limited to IgG1, IgG2, IgG3 or IgG4. In some embodiments, the Fc regions of the first and second antibodies are both of the IgG1 isotype. In some embodiments, the Fc regions of the first and second antibodies are both of the IgG4 isotype. In some embodiments, one of the Fc regions of the antibody is of the IgG1 isotype and the other is of the IgG4 isotype. In a later embodiment, the resulting bispecific antibody includes an Fc region of an IgG1 and an Fc region of an IgG4 and thus may have interesting intermediate properties with respect to activation of effector functions.

The term “knob-into-hole structure” refers to the mutation of the hydrophobic amino acids of CH3 of the antibody Fc. A side-chain amino acid of CH3 of one chain is mutated into a larger hydrophobic amino acid molecule (knob) to enhance the hydrophobic force. Another side-chain amino acid of CH3 is mutated into a smaller amino acid (hole) to reduce steric hindrance. After the mutations, the CH3 with the knob and hole forms a knob-into-hole structure (KiH) in a hydrophobic manner, which facilitates the formation of a heavy chain heterodimer. The KiH mutations mainly occur at inner hydrophobic amino acids of the spatial structure of the CH3 domain. After the mutations, the exposed amino acids on the outside are barely changed; therefore, the effector function of Fc and the resulting immunogenicity are not affected. The term “knob-Fc” refers to the inclusion of a point mutation of T366W in the Fc region of an antibody to form a knob-like spatial structure. Correspondingly, “hole-Fc” refers to the inclusion of point mutations of T366S, L368A and Y407V in the Fc region of an antibody to form a hole-like spatial structure. Point mutations of S354C and Y349C can further be introduced into knob-Fc and hole-Fc respectively to further promote the formation of heterodimers via disulfide bonds. At the same time, point mutations of H435R and Y436F can further be introduced into hole-Fc to reduce the binding to protein A.

In the context of the bispecific antibody of the present disclosure, the Fc region may include one or more amino acid alterations (e.g., insertions, deletions or substitutions), as compared to a specified chimeric form of the Fc region, without altering the desired functionality. For example, the present disclosure includes bispecific antigen-binding molecules including one or more modifications in the Fc region that result in a modified Fc region having a modified binding interaction (e.g., enhanced or attenuated) between Fc and FcRn. Non-limiting examples of such Fc modifications include, for example, the serine (“S”)-to-proline (“P”) mutation at position 228 of the amino acid sequence of a human IgG4 Fc region.

Antibody Expression

In yet another embodiment, the present disclosure provides a polynucleotide encoding the bispecific antibody described herein. The polynucleotide can include a polynucleotide encoding an amino acid sequence of the light chain variable region and/or heavy chain variable region of the antibody, or a polynucleotide encoding an amino acid sequence of the light chain and/or heavy chain of the antibody.

In yet another embodiment, the present disclosure provides an expression vector including the polynucleotide described herein; In one embodiment, the vector is a eukaryotic expression vector. In some embodiments, the polynucleotide described herein is included in one or more expression vectors.

In yet another embodiment, the present disclosure provides a host cell including the polynucleotide described herein or the expression vector described herein; In one embodiment, the host cell is a eukaryotic cell; In one embodiment, the host cell is a mammalian cell.

In yet another embodiment, the present disclosure provides a method for preparing the bispecific antibody described herein, the method including expressing the antibody in the host cell described herein under conditions suitable for the expression of the antibody and collecting the expressed antibody from the host cell.

The present disclosure provides a mammalian host cell used for expressing the recombinant antibody of the present disclosure, which includes a number of immortalized cell lines available from American Type Culture Collection (ATCC). Those cell lines include, in particular, Chinese hamster ovary (CHO) cells, NS0, SP2/0 cells, HeLa cells, baby hamster kidney (BHK) cells, monkey kidney cells (COS), human hepatocellular carcinoma cells, A549 cells, 293T cells and many other cell lines. Mammalian host cells include human, mouse, rat, dog, monkey, pig, goat, cow, horse and hamster cells. Particularly preferred cell lines are selected by determining which cell line has high expression level.

In one embodiment, the present disclosure provides a method for preparing the bispecific antibody described herein, and the method includes introducing an expression vector into a mammalian host cell, and culturing the host cell for a period of time sufficient to allow antibody expression in the host cell or, in one embodiment, antibody secretion into the medium in which the host cell is grown to produce the antibody. The antibody can be isolated from the medium using standard protein purification methods.

It is likely that antibodies expressed by different cell lines or in transgenic animals have different glycosylations from each other. However, all antibodies encoded by the nucleic acid molecules provided herein or including the amino acid sequences provided herein are integral parts of the present disclosure, regardless of the glycosylation of the antibody. Likewise, in some embodiments, nonfucosylated antibodies are advantageous because they generally have more potent efficacy in vitro and in vivo than their fucosylated counterparts, and are unlikely to be immunogenic because their glycan structures are normal components of natural human serum IgG.

Pharmaceutical Composition and Pharmaceutical Formulation

In yet another embodiment, the present disclosure provides a pharmaceutical composition including the bispecific antibody, polynucleotide, expression vector or host cell according to any one of the embodiments herein, and a pharmaceutically acceptable carrier or excipient.

It should be understood that the pharmaceutical composition described herein can be integrated into a suitable carrier, an excipient and other reagents in a formulation for administration in combination, thus providing improved transfer, delivery, tolerance, etc.

The term “pharmaceutical composition” refers to a formulation that allows the biological activity of active ingredients included therein to be present in an effective form and does not include additional ingredients having toxicity unacceptable to a subject to which the formulation is administered.

The pharmaceutical formulation including the antibody described herein, preferably in the form of an aqueous solution or a lyophilized formulation, may be prepared by mixing the bispecific antibody described herein having the desired purity with one or more optional pharmaceutical excipients (Remington's Pharmaceutical Sciences, 16th edition, Osol, A. Ed. (1980)).

The pharmaceutical composition or formulation of the present disclosure can further include one or more additional active ingredients which are required for a specific indication being treated, preferably active ingredients having complementary activities that do not adversely affect one another. In some embodiments, the additional active ingredients are chemotherapeutic agents, immune checkpoint inhibitors, growth inhibitors, antibiotics or various known anti-tumor or anti-cancer agents, which are suitably present in combination in amounts that are effective for the purpose intended.

In some embodiments, the pharmaceutical composition of the present disclosure further includes a composition of a polynucleotide encoding the antibody described herein.

In yet another embodiment, the present disclosure provides a pharmaceutical combination including the bispecific antibody, polynucleotide, expression vector, host cell or pharmaceutical composition according to any one of the embodiments herein, and one or more additional therapeutic agents.

In yet another embodiment, the present disclosure provides a kit including the bispecific antibody, polynucleotide, expression vector, host cell or pharmaceutical composition according to any one of the embodiments herein.

Medical Use

In yet another embodiment, the present disclosure provides use of the bispecific antibody, polynucleotide, expression vector, host cell or pharmaceutical composition according to any one of the embodiments herein in the manufacture of a medicament for preventing or treating cancer.

In yet another embodiment, the present disclosure provides the bispecific antibody, polynucleotide, expression vector, host cell or pharmaceutical composition according to any one of the embodiments herein for use in the prevention or treatment of cancer.

In yet another embodiment, the present disclosure provides a method for preventing or treating cancer including administering to a subject in need thereof the bispecific antibody, polynucleotide, expression vector, host cell or pharmaceutical composition according to any one of the embodiments herein.

In some embodiments, the cancer described in the present disclosure is associated with CD112R and/or TIGIT; In one embodiment, the cancer is selected from melanoma, renal cancer, prostate cancer, breast cancer, colon cancer, lung cancer, bone cancer, pancreatic cancer, skin cancer, head and neck cancer, uterine cancer, ovarian cancer and rectal cancer.

In some embodiments, the routes of administration of the present disclosure include, but are not limited to, oral administration, intravenous administration, subcutaneous administration, intramuscular administration, intra-arterial administration, intra-articular administration (e.g., in arthritic joints), administration by inhalation or aerosol delivery, intratumoral administration, and the like.

In some embodiments, the present disclosure further includes co-administering to a subject a therapeutically effective amount of one or more therapies (e.g., therapeutic modalities and/or additional therapeutic agents). In some embodiments, the therapies include surgical treatment and/or radiation therapy.

In some embodiments, the methods or uses provided herein also include administering to the individual one or more therapies (e.g., therapeutic modalities and/or additional therapeutic agents). The antibody of the present disclosure can be used alone or used in combination with additional therapeutic agents in a therapy. For example, the antibody may be co-administered with at least one additional therapeutic agent, for example, a PD-1 antibody, a PD-L1 antibody, a LAG-3 antibody and/or a CTLA-4 antibody.

Methods for Diagnosis and Detection

In yet another embodiment, the present disclosure provides a method for detecting the presence of CD112R and/or TIGIT in a sample using the bispecific antibody described herein. The term “detection” as used herein includes quantitative or qualitative detection. In some embodiments, the sample is a biological sample. In some embodiments, the biological sample is blood, serum, or other liquid samples of biological origin. In some embodiments, the biological sample includes cells or tissues. The method includes contacting the bispecific antibody of the present disclosure with the sample and detecting the presence of and/or determining the amount of a conjugate that the bispecific antibody and CD112R and/or TIGIT form.

The present disclosure includes any combinations of some embodiments described. Further embodiments of the present disclosure and the full scope of applicability will become apparent from the detailed description provided below. However, it should be understood that the detailed description and the specific examples, while indicating preferred embodiments of the present disclosure, are provided by way of illustration only, as various changes and modifications within the embodiments of the present disclosure will become apparent from the detailed description. All publications, patents and patent applications cited herein, including the citations, are hereby incorporated by reference in their entirety for all purposes.

### EXAMPLES

The following examples are provided to demonstrate and further illustrate some embodiments of the present disclosure and should not be construed as limiting the scope of the present disclosure.

### Example 1. Construction of Expression Vectors for Antibody Molecules

The coding sequences for the heavy chain constant region IgG4 and the light chain constant region κ were cloned from human B lymphocytes (from Beijing Blood Institute) and introduced into the pTT5 plasmid to form vectors HXT4S and HXT2, respectively.

Anti-TIGIT Antibody

Light chain of anti-TIGIT antibody: The DNA sequence encoding hu20-2O18LC-2 was synthesized by Genscript Biotech Ltd. (hereinafter referred to as Genscript for short), digested with BSPQI and ligated to the HXT2 vector (a vector engineered in-house by Junshi Biosciences, derived from pTT5) to obtain the expression vector HXT2-hu20-2O18LC-2.

Heavy chain of anti-TIGIT antibody: The DNA sequence encoding hu20-2O18HC-3 was synthesized by Genscript, digested with BSPQI and ligated to the HXT4s vector (a vector engineered in-house by Junshi Biosciences, derived from pTT5) to obtain the expression vector HXT4s-hu20-2O18HC-3.

Anti-CD112R Antibody

Light chain of anti-CD112R antibody: The DNA sequence encoding hu52-6A10LC-2 was synthesized by Genscript, digested with BSPQI and ligated to the HXT2 vector (a vector engineered in-house by Junshi Biosciences, derived from pTT5) to obtain the expression vector HXT2-hu52-6A10LC-2.

Heavy chain of anti-CD112R antibody: The DNA sequence encoding hu52-7C2HC-1 was synthesized by Genscript, digested with BSPQI and ligated to the HXT4s vector (a vector engineered in-house by Junshi Biosciences, derived from pTT5) to obtain the expression vector HXT4s-hu52-7C2HC-1.

Construction of Expression Vectors for Bispecific Antibody Molecules

Bispecific Antibody Molecule JS-1 (IgG4):

The DNA sequence encoding hu20-2O18LC-2 was synthesized by Genscript, digested with BSPQI and ligated to the HXT2 vector (a vector engineered in-house by Junshi Biosciences, derived from pTT5) to obtain the first expression vector HXT2-hu20-2O18LC-2. IgG4-hole and IgG4-knob were separately synthesized by Genscript, digested with NheI and NotI and ligated to the HXT4s vector to form the vectors HXT4S-muth and HXT4S-mutb. The plasmids HXT4s-hu20-2O18HC-3 and HXT4S-mutb were digested with NheI and NotI respectively to obtain a vector and a fragment, and the vector and the fragment were ligated using T4 DNA ligase to obtain the second expression vector HXT4s-hu20-2O18HC-3 Mut b. The signal peptide and the DNA sequence encoding hu52-52ScFv were synthesized by Genscript, and the EcoRI and ApaI enzyme cutting sites were added to both ends. The synthesized fragment was inserted into HXT4S-muth through EcoRI and ApaI to obtain the third expression vector HX4-hu52-52ScFv Mut h.

Bispecific Antibody Molecule JS-3 (IgG4):

The ScFabhu20 fragment was synthesized in puc57 by Genscript, digested with EcoRI and NheI and ligated to the HXT4S-mutb vector to obtain the first expression vector HX4-ScFabhu20 Mutb. The ScFabhu52 fragment was synthesized in puc57 by Genscript, digested with EcoRI and NheI and ligated to the HXT4S-muth vector to obtain the second expression vector HX4-ScFabhu52 Muth.

Bispecific Antibody Molecule JS-5 (IgG4):

The DNA sequence encoding hu20-2O18LC-2 was synthesized by Genscript, digested with BSPQI and ligated to the HXT2 vector (a vector engineered in-house by Junshi Biosciences, derived from pTT5) to obtain the first expression vector HXT2-hu20-2O18LC-2. The IgG4-hu52-52ScFV fragment was synthesized in puc57 by Genscript, digested with NheI and NotI and ligated to HXT4s-hu20-2O18HC-3 to obtain the second expression vector HXT4s-hu20-2O18HC-3-hu52-52ScFV.

Bispecific Antibody Molecule JS-9 (IgG4):

The DNA sequence encoding hu20-2O18LC-2 was synthesized by Genscript, digested with BSPQI and ligated to the HXT2 vector (a vector engineered in-house by Junshi Biosciences, derived from pTT5) to obtain the first expression vector HXT2-hu20-2O18LC-2. The hu52 SCFV-hu20 2018HC-3 fragment was synthesized in puc57 by Genscript, digested with EcoRI and NheI and ligated to HXT4s to obtain the second expression vector HXT4S-hu52 SCFV-hu20 2018HC-3.

Bispecific Antibody Molecule JS-1 (IgG1):

The first expression vector was the same as that of JS-1 (IgG4). IgG1-hole and IgG1-knob were separately synthesized by Genscript, digested with NheI and NotI and ligated to the HXT1s vector (a vector engineered in-house by Junshi Biosciences, derived from pTT5) to form the vectors HXT1S-muth and HXT1S-mutb. The plasmids HXT1s-hu20-2O18HC-3 and HXT1S-mutb were digested with NheI and NotI respectively to obtain a vector and a fragment, and the vector and the fragment were ligated using T4 DNA ligase to obtain the second expression vector HXT1s-hu20-2O18HC-3 Mut b. The signal peptide and the DNA sequence encoding hu52-52ScFv were synthesized by Genscript, and the EcoRI and ApaI enzyme cutting sites were added to both ends. The synthesized fragment was inserted into HXT1S-muth through EcoRI and ApaI to obtain the third expression vector HX1-hu52-52ScFv Mut h.

Bispecific Antibody Molecule JS-3 (IgG1):

The ScFabhu20 fragment was synthesized in puc57 by Genscript, digested with EcoRI and NheI and ligated to the HXT1S-mutb vector to obtain the first expression vector HX1-ScFabhu20 Mutb. The ScFabhu52 fragment was synthesized in puc57 by Genscript, digested with EcoRI and NheI and ligated to the HXT1S-muth vector to obtain the second expression vector HX1-ScFabhu52 Muth.

Bispecific Antibody Molecule JS-5 (IgG1):

The first expression vector was the same as that of JS-5 (IgG4). The IgG1-hu52-52ScFV fragment was synthesized in puc57 by Genscript, digested with NheI and NotI and ligated to HXT1s-hu20-2O18HC-3 to obtain the second expression vector HXT1s-hu20-2O18HC-3-hu52-52ScFV.

Bispecific Antibody Molecule JS-9 (IgG1):

The first expression vector was the same as that of JS-9 (IgG4). The hu52 SCFV-hu20 2018HC-3 fragment was synthesized in puc57 by Genscript, digested with EcoRI and NheI and ligated to HXT1s to obtain the second expression vector HXT1S-hu52 SCFV-hu20 2018HC-3.

### Example 2. Expression and Purification of Bispecific Antibody Molecules

2.1. Expression of Bispecific Antibody Molecules

Transient expression and purification of bispecific antibody molecules JS-1, JS-3, JS-5 and JS-9: The plasmids constructed above were extracted in large quantities with an endotoxin-controlling kit before subsequent use for expression by mammalian cells. CHO-K1 cells (engineered at the genomic level so that the cells were suitable for transient expression) being cultured were counted and, when the cell density was 2-6×10⁶/mL, expanded by subculturing in a CD CHO medium. The cells were diluted to a density of 1.5-2.0×10⁶/mL the day before transfection. The next day, transfection was performed when the cell density reached about 3.5×10⁶/mL. One tenth of transfection volume of medium was added first, then 1-2 μg/mL transfection volume of plasmid, and finally 3-14 μg/mL of PEI. They were well mixed and then incubated at room temperature for no more than 5 min. Finally, the transfection mixture was slowly added to the prepared cells, and they were well mixed as the mixture was added. After the transfection was complete, the mixture was cultured in a shaker at 36.5° C. at 120 rmp under 7% CO₂ for 6-10 days. The culture was supplemented once on D1, D3, D5, D7 and D9.

2.2. Purification of Bispecific Antibody Molecules

After the culture of the transfection mixture was complete, the mixture was centrifuged at 8000 rmp for 15 min, and the cell supernatant was collected and subjected to sterile filtration. Purification was performed on an AKTA Avant purifier. A column filled with the Mabselect SURE LX affinity filler was first subjected to CIP with 0.1 M NaOH for 30 min and then equilibrated with 3-5 CVs of PBS before sample loading. After the sample loading was complete, the column was rinsed with an affinity chromatography elution buffer. Finally, the target protein was eluted with a pH 3.6 acetic acid-sodium acetate buffer. The sample was neutralized and adjusted to pH 5.5 with a 1 M Tris buffer. Part of the sample was sent for SEC-HPLC analysis to test the purity of the sample. The remaining sample was subjected to polishing purification. The polishing purification used the cation filler ESHMUNO CPX and the Capto MMC ImpRes filler. The equilibration buffer was a pH 5.5, 50 mM acetic acid-sodium acetate system. The eluent was a pH 5.5, 50 mM acetic acid-sodium acetate+1 M NaCl buffer system. Linear elution was adopted to collect the target protein. The final SEC-HPLC monomer purity could reach 95% or above. The bispecific antibody molecule JS-1 was obtained.

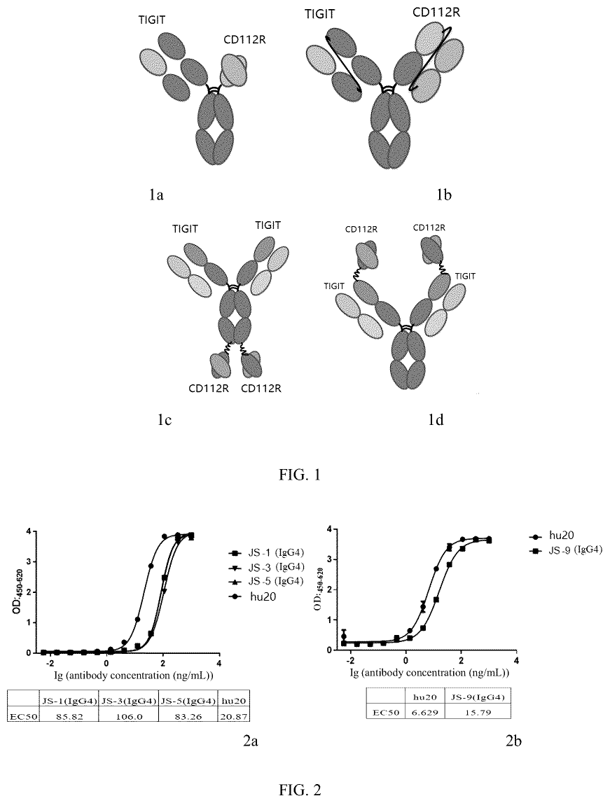

The structures of the bispecific antibody molecules JS-1, JS-3, JS-5 and JS-9 are schematically shown in FIGS. 1a, 1b, 1c and 1d, respectively.

In the bispecific antibody JS-1 (IgG4), the amino acid sequence of the first polypeptide chain (expression vector HXT4s-hu20-2O18HC-3 Mut b) is set forth in SEQ ID NO: 17, the amino acid sequence of the second polypeptide chain (expression vector HXT2-hu20-2O18LC-2) is set forth in SEQ ID NO: 18, and the amino acid sequence of the third polypeptide chain (expression vector HX4-hu52-52ScFv Mut h) is set forth in SEQ ID NO: 19.

(VH is indicated in bold type, and HCDR1-HCDR3 are underlined)
SEQ ID NO: 17
QVQLVQSGAEVKKPGASVKVSCKTSGYAFTEYTMHWVRQAPGKGLEWMG
GINPNTGGTTYNQKFQGRVTLTVDKSSSTAYMELSSLRSEDTVVYYCAKLLRLMY
YFDYWGQGTLVTVSSASTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVESK
YGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISK
AKGQPREPQVYTLPPCQEEMTKNQVSLWCLVKGFYPSDIAVEWESNGQPENNYKTTPP
VLDSDGSFFLYSRLTVDKSRWQEGNVFSCSVMHEALHNHYTQKSLSLSLGK.
(VL is indicated in bold type, and LCDR1-LCDR3 are underlined)
SEQ ID NO: 18
DIQMTQSPSSLSASVGDRVTITCQASQDVRTAVAWYQQKPGKAPKLLIYSASY
RYTGVPSRFSGSGSGTDFTFTISSLQPEDIATYYCHQHYITPWTFGGGTKVEIKRTV
AAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC.
(VL and VH are sequentially indicated in bold type, and LCDR1-LCDR3
and HCDR1-HCDR3 are underlined)
SEQ ID NO: 19
DIVMTQSPDSLAVSLGERATINCKASQSVSNDVAWYQQKPGQPPKLLIYYVSH
RFTGVPDRFSGSGSGTDFTLTISSLQAEDVAVYFCHQAYRSPWTFGCGTKLEIKGG
GGSGGGGSGGGGSGGGGSEVQLVESGGGLVKPGGSLRLSCAASGFTFSSYHMSWV
RQAPGKCLELVSAINSNGINTYYLDTVKGRFTISRDNAKNSLYLQMNSLRAEDTAV
YYCARHEDYYGFAMDYWGQGTLVTVSSGGGGSGGGGSESKYGPPCPPCPAPEFLGG
PSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVCTLPPS
QEEMTKNQVSLSCAVKGFYPSDIAVEWESNGQPENNYKTTPPVLDSDGSFFLVSRLTV
DKSRWQEGNVFSCSVMHEALHNRFTQKSLSLSLGK.

In the bispecific antibody JS-1 (IgG1), the amino acid sequence of the first polypeptide chain (expression vector HXT1s-hu20-2O18HC-3 Mut b) is set forth in SEQ ID NO: 20, the amino acid sequence of the second polypeptide chain (expression vector HXT2-hu20-2O18LC-2) is set forth in SEQ ID NO: 18, and the amino acid sequence of the third polypeptide chain (expression vector HX1-hu52-52ScFv Mut h) is set forth in SEQ ID NO: 21.

(VH is indicated in bold type, and HCDR1-HCDR3 are underlined)
SEQ ID NO: 20
QVQLVQSGAEVKKPGASVKVSCKTSGYAFTEYTMHWVRQAPGKGLEWMG
GINPNTGGTTYNQKFQGRVTLTVDKSSSTAYMELSSLRSEDTVVYYCAKLLRLMY
YFDYWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRVEPKS
CDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKT
ISKAKGQPREPQVYTLPPCREEMTKNQVSLWCLVKGFYPSDIAVEWESNGQPENNYKT
TPPVLDSDGSFFLYSKLTVDKSRWQQGNVFSCSVMHEALHNHYTQKSLSLSPGK.
(VL and VH are sequentially indicated in bold type, and LCDR1-LCDR3
and HCDR1-HCDR3 are underlined)
SEQ ID NO: 21
DIVMTQSPDSLAVSLGERATINCKASQSVSNDVAWYQQKPGQPPKLLIYYVSH
RFTGVPDRFSGSGSGTDFTLTISSLQAEDVAVYFCHQAYRSPWTFGCGTKLEIKGG
GGSGGGGSGGGGSGGGGSEVQLVESGGGLVKPGGSLRLSCAASGFTFSSYHMSWV
RQAPGKCLELVSAINSNGINTYYLDTVKGRFTISRDNAKNSLYLQMNSLRAEDTAV
YYCARHEDYYGFAMDYWGQGTLVTVSSGGGGSGGGGSDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVCTLPPS
REEMTKNQVSLSCAVKGFYPSDIAVEWESNGQPENNYKTTPPVLDSDGSFFLVSKLTV
DKSRWQQGNVFSCSVMHEALHNRFTQKSLSLSPGK.

In the bispecific antibody JS-3 (IgG4), the amino acid sequence of the first polypeptide chain (expression vector HX4-ScFabhu20 Mutb) is set forth in SEQ ID NO: 22, and the amino acid sequence of the second polypeptide chain (expression vector HX4-ScFabhu52 Muth) is set forth in SEQ ID NO: 23.

(VL and VH are sequentially indicated in bold type, and LCDR1-LCDR3
and HCDR1-HCDR3 are underlined)
SEQ ID NO: 22
DIQMTQSPSSLSASVGDRVTITCQASQDVRTAVAWYQQKPGKAPKLLIYSASY
RYTGVPSRFSGSGSGTDFTFTISSLQPEDIATYYCHQHYITPWTFGGGTKVEIKRTV
AAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGECSGGGSGGGSEGG
GSEGGGSEGGGSEGGGSGGGSGQVQLVQSGAEVKKPGASVKVSCKTSGYAFTEYT
MHWVRQAPGKGLEWMGGINPNTGGTTYNQKFQGRVTLTVDKSSSTAYMELSSL
RSEDTVVYYCAKLLRLMYYFDYWGQGTLVTVSSASTKGPSVFPLAPCSRSTSESTAA
LGCLVKDYFPEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMISRTPEVTCV
VVDVSQEDPEVQFNWYVDGVEVHNAKTKPREEQFNSTYRVVSVLTVLHQDWLNGKE
YKCKVSNKGLPSSIEKTISKAKGQPREPQVYTLPPCQEEMTKNQVSLWCLVKGFYPSDI
AVEWESNGQPENNYKTTPPVLDSDGSFFLYSRLTVDKSRWQEGNVFSCSVMHEALHN
HYTQKSLSLSLGK.
(VL and VH are sequentially indicated in bold type, and LCDR1-LCDR3
and HCDR1-HCDR3 are underlined)
SEQ ID NO: 23
DIVMTQSPDSLAVSLGERATINCKASQSVSNDVAWYQQKPGQPPKLLIYYVSH
RFTGVPDRFSGSGSGTDFTLTISSLQAEDVAVYFCHQAYRSPWTFGQGTKLEIKRT
VAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGECSGGGSGGGSEG
GGSEGGGSEGGGSEGGGSGGGSGEVQLVESGGGLVKPGGSLRLSCAASGFTFSSYH
MSWVRQAPGKGLELVSAINSNGINTYYLDTVKGRFTISRDNAKNSLYLQMNSLRA
EDTAVYYCARHEDYYGFAMDYWGQGTLVTVSSASTKGPSVFPLAPCSRSTSESTAA
LGCLVKDYFPEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMISRTPEVTCV
VVDVSQEDPEVQFNWYVDGVEVHNAKTKPREEQFNSTYRVVSVLTVLHQDWLNGKE
YKCKVSNKGLPSSIEKTISKAKGQPREPQVCTLPPSQEEMTKNQVSLSCAVKGFYPSDIA
VEWESNGQPENNYKTTPPVLDSDGSFFLVSRLTVDKSRWQEGNVFSCSVMHEALHNR
FTQKSLSLSLGK.

In the bispecific antibody JS-3 (IgG1), the amino acid sequence of the first polypeptide chain (expression vector HX1-ScFabhu20 Mutb) is set forth in SEQ ID NO: 24, and the amino acid sequence of the second polypeptide chain (expression vector HX1-ScFabhu52 Muth) is set forth in SEQ ID NO: 25.

(VL and VH are sequentially indicated in bold type, and LCDR1-LCDR3
and HCDR1-HCDR3 are underlined)
SEQ ID NO: 24
DIQMTQSPSSLSASVGDRVTITCQASQDVRTAVAWYQQKPGKAPKLLIYSASY
RYTGVPSRFSGSGSGTDFTFTISSLQPEDIATYYCHQHYITPWTFGGGTKVEIKRTV
AAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGECSGGGSGGGSEGG
GSEGGGSEGGGSEGGGSGGGSGQVQLVQSGAEVKKPGASVKVSCKTSGYAFTEYT
MHWVRQAPGKGLEWMGGINPNTGGTTYNQKFQGRVTLTVDKSSSTAYMELSSL
RSEDTVVYYCAKLLRLMYYFDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPCREEMTKNQVSLWCLVKGF
YPSDIAVEWESNGQPENNYKTTPPVLDSDGSFFLYSKLTVDKSRWQQGNVFSCSVMHE
ALHNHYTQKSLSLSPGK.
(VL and VH are sequentially indicated in bold type, and LCDR1-LCDR3
and HCDR1-HCDR3 are underlined)
SEQ ID NO: 25
DIVMTQSPDSLAVSLGERATINCKASQSVSNDVAWYQQKPGQPPKLLIYYVSH
RFTGVPDRFSGSGSGTDFTLTISSLQAEDVAVYFCHQAYRSPWTFGQGTKLEIKRT
VAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGECSGGGSGGGSEG
GGSEGGGSEGGGSEGGGSGGGSGEVQLVESGGGLVKPGGSLRLSCAASGFTFSSYH
MSWVRQAPGKGLELVSAINSNGINTYYLDTVKGRFTISRDNAKNSLYLQMNSLRA
EDTAVYYCARHEDYYGFAMDYWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLN
GKEYKCKVSNKALPAPIEKTISKAKGQPREPQVCTLPPSREEMTKNQVSLSCAVKGFYP
SDIAVEWESNGQPENNYKTTPPVLDSDGSFFLVSKLTVDKSRWQQGNVFSCSVMHEAL
HNRFTQKSLSLSPGK.

In the bispecific antibody JS-5 (IgG4), the amino acid sequence of the first polypeptide chain (expression vector HXT4s-hu20-2O18HC-3-hu52-52ScFV) is set forth in SEQ ID NO: 26, and the amino acid sequence of the second polypeptide chain (expression vector HXT2-hu20-2O18LC-2) is set forth in SEQ ID NO: 18.

(VL and VH are sequentially indicated in bold type, and LCDR1-LCDR3
and HCDR1-HCDR3 are underlined)
SEQ ID NO: 26
QVQLVQSGAEVKKPGASVKVSCKTSGYAFTEYTMHWVRQAPGKGLEWMG
GINPNTGGTTYNQKFQGRVTLTVDKSSSTAYMELSSLRSEDTVVYYCAKLLRLMY
YFDYWGQGTLVTVSSASTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVESK
YGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGGGGSGGG
GSDIVMTQSPDSLAVSLGERATINCKASQSVSNDVAWYQQKPGQPPKLLIYYVSHR
FTGVPDRFSGSGSGTDFTLTISSLQAEDVAVYFCHQAYRSPWTFGCGTKLEIKGGG
GSGGGGSGGGGSGGGGSEVQLVESGGGLVKPGGSLRLSCAASGFTFSSYHMSWVR
QAPGKCLELVSAINSNGINTYYLDTVKGRFTISRDNAKNSLYLQMNSLRAEDTAVY
YCARHEDYYGFAMDYWGQGTLVTVSS.

In the bispecific antibody JS-5 (IgG1), the amino acid sequence of the first polypeptide chain (expression vector HXT1s-hu20-2O18HC-3-hu52-52ScFV) is set forth in SEQ ID NO: 27, and the amino acid sequence of the second polypeptide chain (expression vector HXT2-hu20-2O18LC-2) is set forth in SEQ ID NO: 18.

(VL and VH are sequentially indicated in bold type, and LCDR1-LCDR3
and HCDR1-HCDR3 are underlined)
SEQ ID NO: 27
QVQLVQSGAEVKKPGASVKVSCKTSGYAFTEYTMHWVRQAPGKGLEWMG
GINPNTGGTTYNQKFQGRVTLTVDKSSSTAYMELSSLRSEDTVVYYCAKLLRLMY
YFDYWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGGGGSGG
GGSDIVMTQSPDSLAVSLGERATINCKASQSVSNDVAWYQQKPGQPPKLLIYYVSH
RFTGVPDRFSGSGSGTDFTLTISSLQAEDVAVYFCHQAYRSPWTFGCGTKLEIKGG
GGSGGGGSGGGGSGGGGSEVQLVESGGGLVKPGGSLRLSCAASGFTFSSYHMSWV
RQAPGKCLELVSAINSNGINTYYLDTVKGRFTISRDNAKNSLYLQMNSLRAEDTAV
YYCARHEDYYGFAMDYWGQGTLVTVSS.

In the bispecific antibody JS-9 (IgG4), the amino acid sequence of the first polypeptide chain (expression vector HXT4S-hu52 SCFV-hu20 2018HC-3) is set forth in SEQ ID NO: 28, and the amino acid sequence of the second polypeptide chain (expression vector HXT2-hu20-2018LC-2) is set forth in SEQ ID NO: 18.

(VL and VH are sequentially indicated in bold type, and LCDR1-LCDR3
and HCDR1-HCDR3 are underlined)
SEQ ID NO: 28
DIVMTQSPDSLAVSLGERATINCKASQSVSNDVAWYQQKPGQPPKLLIYYVSH
RFTGVPDRFSGSGSGTDFTLTISSLQAEDVAVYFCHQAYRSPWTFGCGTKLEIKGG
GGSGGGGSGGGGSGGGGSEVQLVESGGGLVKPGGSLRLSCAASGFTFSSYHMSWV
RQAPGKCLELVSAINSNGINTYYLDTVKGRFTISRDNAKNSLYLQMNSLRAEDTAV
YYCARHEDYYGFAMDYWGQGTLVTVSSGGGGSGGGGSQVQLVQSGAEVKKPGA
SVKVSCKTSGYAFTEYTMHWVRQAPGKGLEWMGGINPNTGGTTYNQKFQGRVT
LTVDKSSSTAYMELSSLRSEDTVVYYCAKLLRLMYYFDYWGQGTLVTVSSASTKG
PSVFPLAPCSRSTSESTAALGCLVKDYFPEPVTVSWNSGALTSGVHTFPAVLQSSGLYSL
SSVVTVPSSSLGTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQ
EGNVFSCSVMHEALHNHYTQKSLSLSLGK.

In the bispecific antibody JS-9 (IgG1), the amino acid sequence of the first polypeptide chain (expression vector HXT1S-hu52 SCFV-hu20 2018HC-3) is set forth in SEQ ID NO: 29, and the amino acid sequence of the second polypeptide chain (expression vector HXT2-hu20-2018LC-2) is set forth in SEQ ID NO: 18.

(VL and VH are sequentially indicated in bold type, and LCDR1-LCDR3
and HCDR1-HCDR3 are underlined)
SEQ ID NO: 29
DIVMTQSPDSLAVSLGERATINCKASQSVSNDVAWYQQKPGQPPKLLIYYVSH
RFTGVPDRFSGSGSGTDFTLTISSLQAEDVAVYFCHQAYRSPWTFGCGTKLEIKGG
GGSGGGGSGGGGSGGGGSEVQLVESGGGLVKPGGSLRLSCAASGFTFSSYHMSWV
RQAPGKCLELVSAINSNGINTYYLDTVKGRFTISRDNAKNSLYLQMNSLRAEDTAV
YYCARHEDYYGFAMDYWGQGTLVTVSSGGGGSGGGGSQVQLVQSGAEVKKPGA
SVKVSCKTSGYAFTEYTMHWVRQAPGKGLEWMGGINPNTGGTTYNQKFQGRVT
LTVDKSSSTAYMELSSLRSEDTVVYYCAKLLRLMYYFDYWGQGTLVTVSSASTKG
PSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKS
RWQQGNVFSCSVMHEALHNHYTQKSLSLSPGK.

### Example 3. Stability of Bispecific Antibodies

3.1. Thermostability of Bispecific Antibodies

1. Objective

To test the thermostability of the bispecific antibodies of the present disclosure. The stability of the bifunctional proteins in the conventional formulation (20 mM histidine-histidine hydrochloride buffer, 230 mM trehalose, pH 5.5) was tested by DSF (differential scanning fluorimetry).

2. Procedures and Results

The antibody samples were exchanged into the above buffer with the concentration of the samples controlled at about 5 mg/mL, and DSF tests were performed. The results are shown in Table 1. The results show that the bispecific antibodies JS-1 (IgG4), JS-3 (IgG4), JS-5 (IgG1), JS-5 (IgG4) and JS-9 (IgG1) of the present disclosure all have a melting temperature (Tm) of 59.5° C. or higher in the above buffer, which indicates good thermostability.

TABLE 1
Thermostability tests for bispecific antibodies

| Sample | Tm1 (° m) | Tm2 (° m) |
|---|---|---|
| JS-1(IgG4) | 59.5 | / |
| JS-3(IgG4) | 60.4 | 66.2 |
| JS-5(IgG1) | 67.4 | 74.8 |
| JS-5(IgG4) | 64.6 | / |
| JS-9(IgG1) | 61.7 | / |

3.2. High-Temperature Stability of Bispecific Antibodies

1. Objective

To test the stability of the bispecific antibodies at a high temperature.

2. Procedures and Results

The antibody samples were exchanged into a pH 5.5 buffer system (20 mM histidine-histidine hydrochloride buffer, 230 mM trehalose) with the concentration of the samples controlled at about 5 mg/mL, and the buffer was aliquoted into vials at 500 μL/vial. The vials were placed in a 40° C. incubator to test stability at 0 W, 2 W and 4 W. Samples were sent for analysis according to Table 2. Stability was assessed by the following parameters: (a) the content of antibody monomers, aggregates or fragments determined by SEC-HPLC (size exclusion chromatography); (b) the molecular weight of the antibody determined by CE-SDS (sodium dodecyl sulfate capillary electrophoresis); and (c) the bioactivity of the antibody determined by an ELISA or reporter gene assay.

The results show that after sitting at a high temperature of 40° C. for 4 weeks, the bispecific antibodies JS-1 (IgG4), JS-3 (IgG4), JS-5 (IgG1), JS-5 (IgG4) and JS-9 (IgG1) of the present disclosure underwent a significant change in neither purity (SEC-HPLC, R-CE-SDS (reduced electrophoresis), NR-CE-SDS (non-reduced electrophoresis)) nor bioactivity: they have good thermostability. The specific results are shown in Table 2.

TABLE 2
The stability of the bispecific antibodies after sitting at 40° C. for 4 weeks

| | | | | | NR-CE- | R-CE- | Bioactivity | |
|---|---|---|---|---|---|---|---|---|
| | | SEC-HPLC (%) | | | SDS | SDS | TIGIT binding | CD112R binding |
| Sample | Time | Aggregate | Monomer | Fragment | (%) | (%) | activity Elisa (%) | activity Elisa (%) |
| JS- | 0 weeks | 0.5 | 99.0 | 0.5 | 96.7 | 98.0 | 100 | 101 |
| 1(IgG4) | 2 weeks | 0.5 | 97.5 | 2.1 | 96.5 | 98.1 | 75 | 87 |
| | 4 weeks | 0.5 | 96.9 | 2.6 | 94.7 | 95.7 | 97 | 86 |
| JS- | 0 weeks | 2.0 | 97.3 | 0.7 | 99.3 | 99.1 | 111 | 102 |
| 3(IgG4) | 2 weeks | 2.2 | 97.0 | 0.8 | 99.6 | 97.6 | 88 | 98 |
| | 4 weeks | 2.4 | 96.7 | 0.9 | 99.5 | 94.2 | 84 | 88 |
| JS- | 0 weeks | 0.1 | 99.7 | 0.2 | 98.5 | 97.2 | 97 | 99 |
| 5(IgG1) | 2 weeks | 0.8 | 98.7 | 0.6 | 97.4 | 96.3 | 100 | 99 |
| | 4 weeks | 1.2 | 98.0 | 0.8 | 96.5 | 92.6 | 72 | 80 |
| JS- | 0 weeks | 0.5 | 99.5 | ND | 98.6 | 98.5 | 108 | 115 |
| 5(IgG4) | 2 weeks | 1.2 | 98.7 | 0.1 | 96.4 | 97.4 | 89 | 124 |
| | 4 weeks | 1.7 | 98.0 | 0.3 | 94.8 | 97.1 | 103 | 105 |
| JS- | 0 weeks | 0.2 | 98.3 | 1.5 | 91.2 | 98.9 | 105 | 99 |
| 9(IgG1) | 2 weeks | 0.3 | 95.3 | 4.4 | 90.1 | 97.5 | 98 | 95 |
| | 4 weeks | 0.4 | 93.9 | 5.6 | 89.0 | 96.7 | 99 | 98 |

### Example 4. ELISA Assays of Bispecific Antibodies for Binding to Human TIGIT and Human CD112R

4.1. ELISA Assays of Bispecific Antibodies for Binding to Human TIGIT

1. Objective

To assay the bispecific antibodies of the present disclosure for binding to human TIGIT by ELISA.

2. Procedures
a. A 96-well plate was coated with 1.5 μg/mL recombinant human TIGIT (His Tag) (Suzhou Junmeng) as the antigen at 100 μL/well and incubated at 37° C. for 1.5 h.
b. The plate was washed 4 times with 300 μL/well 1×PBST, and 2% BSA was added at 200 μL/well. The plate was blocked at 37° C. for 1.5 h.
c. The plate was washed 4 times with 300 μL/well 1×PBST, and serial dilutions of the bispecific antibodies JS-1,3,5,9 and a negative control: antibody (anti-KLH hIgG4) (initial concentration 1.0 μg/mL, 3-fold serially diluted to 12 concentration points, 100 μL/well) were added. The plate was incubated at 37° C. for 1 h.
d. The plate was washed 4 times with 300 μL/well 1×PBST.
e. 5000-fold diluted horseradish peroxidase (HRP)-conjugated mouse anti-human IgG4 Fc antibody (Southern Biotech, 9200-05) was added at 100 μL/well, and the plate was incubated at 37° C. for 1 h.
f. The plate was washed 4 times with 300 μL/well 1×PBST.
g. 0.1 mg/mL TMB was added at 100 μL/well, and after the plate was incubated at 37° C. for 15 min, a 2 M solution of hydrochloric acid was added at 100 μL/well to stop the reactions.
h. The absorbance was measured at 450 nm/620 nm on a microplate reader, and data were analyzed using Graphpad Prism7.

3. Results

The results are shown in FIG. 2. The data show that JS-1 (IgG4), JS-3 (IgG4), JS-5 (IgG4) and JS-9 (IgG4) all have reduced binding activity to human TIGIT compared to hu20 mAb but still maintain relatively high binding affinity.

4.2. ELISA Assays of Bispecific Antibodies for Binding to Human CD112R

1. Objective

To assay the bispecific antibodies of the present disclosure for binding to human CD112R by ELISA.

2. Procedures
a. A 96-well plate was coated with 1.0 μg/mL recombinant human HX1 hCD112R Fc (Suzhou Junmeng) as the antigen at 100 μL/well and incubated at 37° C. for 1.5 h.
b. The plate was washed 4 times with 300 μL/well 1×PBST, and 2% BSA was added at 200 μL/well. The plate was blocked at 37° C. for 1.5 h.
c. The plate was washed 4 times with 300 μL/well 1×PBST, and serial dilutions of the bispecific antibodies JS-1,3,5,9 and a negative control: antibody (anti-KLH hIgG4) (initial concentration 3.0 μg/mL, 3-fold serially diluted to 12 concentration points, 100 μL/well) were added. The plate was incubated at 37° C. for 1 h.
d. The plate was washed 4 times with 300 μL/well 1×PBST.
e. 5000-fold diluted horseradish peroxidase (HRP)-conjugated mouse anti-human IgG4 Fc antibody (Southern Biotech, 9200-05) was added at 100 μL/well, and the plate was incubated at 37° C. for 1 h.
f. The plate was washed 4 times with 300 μL/well 1×PBST.
g. 0.1 mg/mL TMB was added at 100 μL/well, and after the plate was incubated at 37° C. for 15 min, a 2 M solution of hydrochloric acid was added at 100 μL/well to stop the reactions.
h. The absorbance was measured at 450 nm/620 nm on a microplate reader, and data were analyzed using Graphpad Prism7.

3. Results

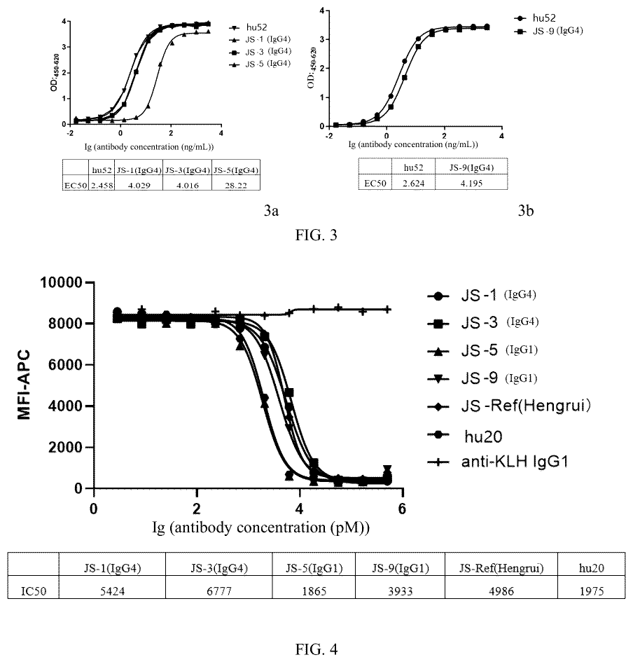

The results are shown in FIG. 3. The data show that JS-1 (IgG4), JS-3 (IgG4), JS-5 (IgG4) and JS-9 (IgG4) all have reduced binding activity to human CD112R compared to hu52 mAb but still maintain relatively high binding affinity.

### Example 5. Blocking Activity of Bispecific Antibodies Against TIGIT/PVR Interaction

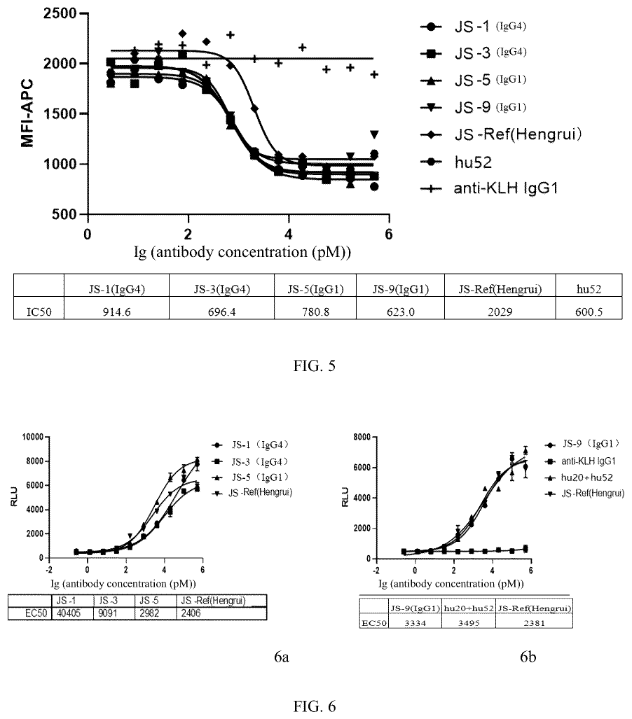

Jurkat-hTIGIT cells (human T lymphocytes, human TIGIT positive) were incubated with different concentrations of the bispecific antibodies JS-1, JS-3, JS-5 and JS-9 and 1 μg/mL human PVR-mFc protein at 4° C. for 30 min, then washed and incubated with a fluorescence-labeled anti-mouse IgG secondary antibody (1:200) in a dark place at 4° C. for 30 min. Finally, the cells were collected using a flow cytometer (BD Canto II), and the fluorescent antibodies bound to the cell surface were detected. Raw data were analyzed using FlowJo to obtain MFI values, antibody dose-dependent binding curves (FIG. 4) were fitted using GraphPad, and IC50 was calculated. The positive controls were hu20 (a recombinant humanized anti-TIGIT monoclonal antibody) and JS-Ref (Hengrui) (an anti-TIGIT/CD112R bispecific antibody of Hengrui, expressed and purified according to the sequence of the patent WO2021180205), and the negative control was an anti-KLH-hIgG4 antibody.

As shown in FIG. 4, JS-1 (IgG4), JS-3 (IgG4), JS-5 (IgG1), JS-9 (IgG1), JS-Ref (Hengrui) and hu20 can all significantly inhibit the binding of PVR, a ligand for TIGIT, to human TIGIT on the surface of Jurkat cells, with IC₅₀ of 5424 pM, 6777 pM, 1865 pM, 3933 pM, 4986 pM and 1975 pM, respectively.

### Example 6. Blocking Activity of Bispecific Antibodies Against CD112R/CD112 Interaction

CHO hCD112 cells (Chinese hamster ovary cells, human CD112 positive) were incubated with different concentrations of the bispecific antibodies JS-1, JS-3, JS-5 and JS-9 and 1 μg/mL human CD112R-mFc protein at 4° C. for 30 min, then washed twice and incubated with a fluorescence-labeled anti-mouse IgG secondary antibody (1:200) in a dark place at 4° C. for 30 min. Finally, the cells were collected using a flow cytometer (BD Canto II), and the fluorescent antibodies bound to the cell surface were detected. Raw data were analyzed using FlowJo to obtain MFI values, antibody dose-dependent binding curves (FIG. 4) were fitted using GraphPad, and IC50 was calculated. The positive controls were hu52 (a recombinant humanized anti-CD112R monoclonal antibody) and JS-Ref (Hengrui) (an anti-TIGIT/CD112R bispecific antibody of Hengrui, expressed and purified according to the sequence of the patent WO2021180205), and the negative control was an anti-KLH-hIgG1 antibody.

As shown in FIG. 5, JS-1 (IgG4), JS-3 (IgG4), JS-5 (IgG1), JS-9 (IgG1), JS-Ref (Hengrui) and hu52 can all significantly inhibit the binding of the human CD112R protein to human CD112 on the surface of CHO cells, with IC50 of 914.6 pM, 696.4 pM, 780.8 pM, 623.0 pM, 2029 pM and 600.5 pM, respectively.

### Example 7. Activity of Bispecific Antibodies in CD112/TIGIT, CD155/TIGIT and CD112/CD112R Luciferase Reporter Gene System

The target cells, CHO CD112 CD155 (stably expressing human CD155, CD112 and CD3scFv) cells, were seeded into a 96-well flat-bottom white plate at 5×10⁴ cells/well and left overnight. The next day, after the media in the cell wells was pipetted off, different concentrations of the JS antibodies or control antibodies (initial concentration 500 nM, 5-fold dilution, 10 concentration gradients in total) were added to the target cells with an experimental buffer (RPMI 1640 (1×)+2% FBS), and the plate was incubated at 37° C. for 30 min. Effector cells Jurkat CD112R TIGIT (stably expressing human CD112R, human TIGIT and luc2P/NFAT-RE) were then added to the cell plate at 1×10⁵ cells/well and co-incubated in a 37° C. incubator for 6 h. Finally, the ONE-Glo luciferase assay reagent (Vazyme) was added to the mixed systems of cells and antibodies, and chemiluminescence signals were detected using a multi-mode microplate reader (TECAN M1000 pro). A four-parameter regression curve was fitted by using GraphPad Prism software and EC₅₀ values were calculated. The positive controls were hu52 (recombinant humanized anti-CD112R monoclonal antibody), hu20 (recombinant humanized anti-TIGIT monoclonal antibody) and JS-Ref (Hengrui) (an anti-TIGIT/CD112R bispecific antibody of Hengrui, in-house expressed and purified according to the sequence of the patent WO2021180205), and the negative control was an anti-KLH-hIgG1 antibody.

As shown in FIG. 6, JS-1 (IgG4), JS-3 (IgG4), JS-5 (IgG1), JS-9 (IgG1), JS-Ref (Hengrui) and hu20+hu52 have strong T cell activating activity in the above reporter gene system: the EC₅₀ values were 40405 pM, 9091 pM, 2982 pM, 3334 pM, 2381 pM and 3495 pM, respectively; JS-5 (IgG1) and JS-9 (IgG1) have comparable activity to the hu20+hu52 combination group and JS-Ref (Hengrui).

### Example 8. Tumor-Inhibiting Effects of Bispecific Antibodies in Mouse Model of Liver Cancer Built by Transplanting H22-hPDL1 Subcutaneously to BALB/c-hPD-1/hPDL1/hTIGIT/hPVRIG Humanized Mice

1×10⁶ H22-hPDL1 cells were inoculated subcutaneously into the right side of BALB/c-hPD-1/hPDL1/hTIGIT/hPVRIG humanized mice (Jiangsu Gempharmatech Co., Ltd.) at 0.1 mL/mouse. When the mean tumor volume was about 76 mm 3, 42 animals were selected by tumor volume and randomized into 7 groups of 6:
normal saline control;
hu20, 22.5 mg/kg;
hu52, 22.5 mg/kg;
hu20+hu52, 22.5+22.5 mg/kg;
JS-5 (IgG1), 10 mg/kg;
JS-5 (IgG1), 30 mg/kg;
JS-Ref (Hengrui), 30 mg/kg;

Administration was performed on the day of grouping, and the route of administration for all the groups was intraperitoneal injection. Administration was performed twice a week, and was continuously performed 6 times. The experiment ended 4 days after the last administration. Tumor volume and body weight of mice were measured and recorded 3 times a week. At the end of the experiment, mice were euthanized and tumor inhibition TGI_{TV} was calculated.
Calculation formula for TGI_{TV}: RTVn=Vₙₜ/Vₙ₀, TGI_{TV}=(1−mean RTVₜᵣₑₐₜ/mean RTV_{vehicle})×100%
Vₙₜ: tumor volume of mice n on day t;
Vₙ₀: tumor volume of mice n on day 0;
RTVn: relative tumor volume of mice n on day t;
mean RTVₜᵣₑₐₜ: mean RTV of a treatment group;
mean RTV_{vehicle}: mean RTV of a vehicle group.

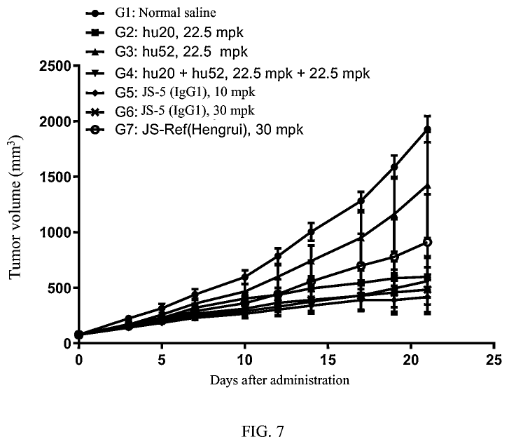

As shown in FIG. 7, at the end of the experiment, the mean tumor volume of the normal saline control group was 1927.54 mm³. Hu20 showed a significant tumor-inhibiting effect when administered at the dose of 22.5 mg/kg, leading to a mean tumor volume of 598.19 mm 3 and 69.26% TGI (p<0.001); Hu52 led to a mean tumor volume of 1425.27 mm 3 and 26.63% TGI when administered at the dose of 22.5 mg/kg; hu20 (22.5 mg/kg)+hu52 (22.5 mg/kg) showed a significant tumor-inhibiting effect, leading to a mean tumor volume of 558.98 mm³ and 70.59% TGI (p<0.001); JS-5 (IgG1) showed significant tumor-inhibiting effects when administered at the doses of 10 mg/kg and 30 mg/kg, leading to tumor volumes of 416.26 mm³ and 483.71 mm³, respectively, and 79.21% TGI and 75.28% TGI (p<0.001), respectively. JS-Ref (Hengrui) led to a tumor volume of 909.85 mm 3 and 53.86% TGI (p<0.05) when administered at the dose of 30 mg/kg. When administered at the same dose, JS-5 showed a significantly better tumor-inhibiting effect than JS-Ref (Hengrui).

What is claimed is:

1. A bispecific antibody comprising a binding domain that binds to CD112R and a binding domain that binds to TIGIT, wherein the binding domain that binds to CD112R comprises:
HCDR1, HCDR2 and HCDR3 of the amino acid sequence set forth in SEQ ID NO: 1, and/or LCDR1, LCDR2 and LCDR3 of the amino acid sequence set forth in SEQ ID NO: 2;
wherein the HCDR1, HCDR2, HCDR3, LCDR1, LCDR2 and LCDR3 are defined according to the Kabat, IMGT, Chothia, AbM or Contact numbering system.

2. The bispecific antibody according to claim 1, wherein the binding domain that binds to TIGIT comprises:
HCDR1, HCDR2 and HCDR3 of the amino acid sequence set forth in SEQ ID NO: 9, and/or LCDR1, LCDR2 and LCDR3 of the amino acid sequence set forth in SEQ ID NO: 10;
wherein the HCDR1, HCDR2, HCDR3, LCDR1, LCDR2 and LCDR3 are defined according to the Kabat, IMGT, Chothia, AbM or Contact numbering system.

3. The bispecific antibody according to claim 2, wherein the binding domain that binds to CD112R comprises, according to the Kabat numbering system: an HCDR1 whose amino acid sequence is set forth in SEQ ID NO: 3, an HCDR2 whose amino acid sequence is set forth in SEQ ID NO: 4, an HCDR3 whose amino acid sequence is set forth in SEQ ID NO: 5, an LCDR1 whose amino acid sequence is set forth in SEQ ID NO: 6, an LCDR2 whose amino acid sequence is set forth in SEQ ID NO: 7, and an LCDR3 whose amino acid sequence is set forth in SEQ ID NO: 8.

4. The bispecific antibody according to claim 2, wherein the binding domain that binds to TIGIT comprises: an HCDR1 whose amino acid sequence is set forth in SEQ ID NO: 11, an HCDR2 whose amino acid sequence is set forth in SEQ ID NO: 12, an HCDR3 whose amino acid sequence is set forth in SEQ ID NO: 13, an LCDR1 whose amino acid sequence is set forth in SEQ ID NO: 14, an LCDR2 whose amino acid sequence is set forth in SEQ ID NO: 15, and an LCDR3 whose amino acid sequence is set forth in SEQ ID NO: 16.

5. The bispecific antibody according to claim 1, wherein the binding domain that binds to CD112R comprises:
a heavy chain variable region having at least 90%, 91%, 92%, 93%, 94%, 95%, 96%, 97%, 98%, 99% or 100% sequence identity to the amino acid sequence set forth in SEQ ID NO: 1, and a light chain variable region having at least 90%, 91%, 92%, 93%, 94%, 95%, 96%, 97%, 98%, 99% or 100% sequence identity to the amino acid sequence set forth in SEQ ID NO: 2; or
a heavy chain variable region having at least 90%, 91%, 92%, 93%, 94%, 95%, 96%, 97%, 98%, 99% or 100% sequence identity to the amino acid sequence set forth in SEQ ID NO: 30, and a light chain variable region having at least 90%, 91%, 92%, 93%, 94%, 95%, 96%, 97%, 98%, 99% or 100% sequence identity to the amino acid sequence set forth in SEQ ID NO: 31.

6. The bispecific antibody according to claim 1, wherein the binding domain that binds to CD112R comprises: a heavy chain variable region whose amino acid sequence is set forth in SEQ ID NO: 1 and a light chain variable region whose amino acid sequence is set forth in SEQ ID NO: 2; or a heavy chain variable region whose amino acid sequence is set forth in SEQ ID NO: 30 and a light chain variable region whose amino acid sequence is set forth in SEQ ID NO: 31.

7. The bispecific antibody according to claim 2, wherein the binding domain that binds to TIGIT comprises: a heavy chain variable region having at least 90%, 91%, 92%, 93%, 94%, 95%, 96%, 97%, 98%, 99% or 100% sequence identity to the amino acid sequence set forth in SEQ ID NO: 9, and a light chain variable region having at least 90%, 91%, 92%, 93%, 94%, 95%, 96%, 97%, 98%, 99% or 100% sequence identity to the amino acid sequence set forth in SEQ ID NO: 10.

8. The bispecific antibody according to claim 2, wherein the binding domain that binds to TIGIT comprises: a heavy chain variable region whose amino acid sequence is set forth in SEQ ID NO: 9, and a light chain variable region whose amino acid sequence is set forth in SEQ ID NO: 10.

9. The bispecific antibody according to claim 1, wherein the binding domain that binds to CD112R is an ScFab fragment, a Fab fragment, an scFv fragment or an Fv fragment, and the binding domain that binds to TIGIT is an ScFab fragment, a Fab fragment, an scFv fragment or an Fv fragment.

10. The bispecific antibody according to claim 8, further comprising an Fc domain, wherein the Fc domain is an IgG1 Fc domain, an IgG2 Fc domain, an IgG3 Fc domain or an IgG4 Fc domain.

11. The bispecific antibody according to claim 8, wherein the binding domain that binds to TIGIT is linked to the N-terminus of the Fc domain by a hinge region, and the binding domain that binds to CD112R is linked to the N-terminus of the Fc domain by a hinge region or to the C-terminus of the Fc domain by a linking peptide or to the N-terminus of the heavy chain variable region of the binding domain that binds to TIGIT by a linking peptide.

12. The bispecific antibody according to claim 1, wherein the bispecific antibody has:
three polypeptide chains, wherein the first polypeptide chain has, from N-terminus to C-terminus, VH1-CH1-hinge region-first Fc region, the second polypeptide chain has, from N-terminus to C-terminus, VL1-CL, and the third polypeptide chain has, from N-terminus to C-terminus, VL2-linking peptide-VH2-linking peptide-hinge region-second Fc region; In one embodiment, the VH1-CH1 of the first polypeptide chain and the VL1-CL of the second polypeptide chain form the binding domain that binds to TIGIT, and the VL2-linking peptide-VH2 of the third polypeptide chain forms the binding domain that binds to CD112R; or
two polypeptide chains, wherein the first polypeptide chain has, from N-terminus to C-terminus, VL1-CL-linking peptide-VH1-CH1-hinge region-first Fc region, and the second polypeptide chain has, from N-terminus to C-terminus, VL2-CL-linking peptide-VH2-CH1-hinge region-second Fc region; In one embodiment, the VL1-CL-linking peptide-VH1-CH1 of the first polypeptide chain forms the binding domain that binds to TIGIT, and the VL2-CL-linking peptide-VH2-CH1 of the second polypeptide chain forms the binding domain that binds to CD112R; or
two identical heavy chains and two identical light chains, wherein the heavy chains have, from N-terminus to C-terminus, VH1-CH1-hinge region-Fc region-linking peptide-VL2-linking peptide-VH2 or VL2-linking peptide-VH2-linking peptide-VH1-CH1-hinge region-Fc region, and the light chains have, from N-terminus to C-terminus, VL1-CL; In one embodiment, the VH1-CH1 of the heavy chains and the VL1-CL of the light chains form the binding domain that binds to TIGIT, and the VL2-linking peptide-VH2 of the heavy chains forms the binding domain that binds to CD112R.

13. The bispecific antibody according to claim 1, wherein the bispecific antibody comprises:
a first polypeptide chain having at least 90%, 91%, 92%, 93%, 94%, 95%, 96%, 97%, 98%, 99% or 100% sequence identity to the amino acid sequence set forth in SEQ ID NO: 17, a second polypeptide chain having at least 90%, 91%, 92%, 93%, 94%, 95%, 96%, 97%, 98%, 99% or 100% sequence identity to the amino acid sequence set forth in SEQ ID NO: 18, and a third polypeptide chain having at least 90%, 91%, 92%, 93%, 94%, 95%, 96%, 97%, 98%, 99% or 100% sequence identity to the amino acid sequence set forth in SEQ ID NO: 19; or
a first polypeptide chain having at least 90%, 91%, 92%, 93%, 94%, 95%, 96%, 97%, 98%, 99% or 100% sequence identity to the amino acid sequence set forth in SEQ ID NO: 20, a second polypeptide chain having at least 90%, 91%, 92%, 93%, 94%, 95%, 96%, 97%, 98%, 99% or 100% sequence identity to the amino acid sequence set forth in SEQ ID NO: 18, and a third polypeptide chain having at least 90%, 91%, 92%, 93%, 94%, 95%, 96%, 97%, 98%, 99% or 100% sequence identity to the amino acid sequence set forth in SEQ ID NO: 21; or
a first polypeptide chain having at least 90%, 91%, 92%, 93%, 94%, 95%, 96%, 97%, 98%, 99% or 100% sequence identity to the amino acid sequence set forth in SEQ ID NO: 22, and a second polypeptide chain having at least 90%, 91%, 92%, 93%, 94%, 95%, 96%, 97%, 98%, 99% or 100% sequence identity to the amino acid sequence set forth in SEQ ID NO: 23; or
a first polypeptide chain having at least 90%, 91%, 92%, 93%, 94%, 95%, 96%, 97%, 98%, 99% or 100% sequence identity to the amino acid sequence set forth in SEQ ID NO: 24, and a second polypeptide chain having at least 90%, 91%, 92%, 93%, 94%, 95%, 96%, 97%, 98%, 99% or 100% sequence identity to the amino acid sequence set forth in SEQ ID NO: 25; or
two heavy chains having at least 90%, 91%, 92%, 93%, 94%, 95%, 96%, 97%, 98%, 99% or 100% sequence identity to the amino acid sequence set forth in SEQ ID NO: 26, and two light chains having at least 90%, 91%, 92%, 93%, 94%, 95%, 96%, 97%, 98%, 99% or 100% sequence identity to the amino acid sequence set forth in SEQ ID NO: 18; or
two heavy chains having at least 90%, 91%, 92%, 93%, 94%, 95%, 96%, 97%, 98%, 99% or 100% sequence identity to the amino acid sequence set forth in SEQ ID NO: 27, and two light chains having at least 90%, 91%, 92%, 93%, 94%, 95%, 96%, 97%, 98%, 99% or 100% sequence identity to the amino acid sequence set forth in SEQ ID NO: 18; or
two heavy chains having at least 90%, 91%, 92%, 93%, 94%, 95%, 96%, 97%, 98%, 99% or 100% sequence identity to the amino acid sequence set forth in SEQ ID NO: 28, and two light chains having at least 90%, 91%, 92%, 93%, 94%, 95%, 96%, 97%, 98%, 99% or 100% sequence identity to the amino acid sequence set forth in SEQ ID NO: 18; or
two heavy chains having at least 90%, 91%, 92%, 93%, 94%, 95%, 96%, 97%, 98%, 99% or 100% sequence identity to the amino acid sequence set forth in SEQ ID NO: 29, and two light chains having at least 90%, 91%, 92%, 93%, 94%, 95%, 96%, 97%, 98%, 99% or 100% sequence identity to the amino acid sequence set forth in SEQ ID NO: 18.

14. The bispecific antibody according to claim 1, wherein the bispecific antibody comprises:
a first polypeptide chain whose amino acid sequence is set forth in SEQ ID NO: 17, a second polypeptide chain whose amino acid sequence is set forth in SEQ ID NO: 18, and a third polypeptide chain whose amino acid sequence is set forth in SEQ ID NO: 19; or
a first polypeptide chain whose amino acid sequence is set forth in SEQ ID NO: 20, a second polypeptide chain whose amino acid sequence is set forth in SEQ ID NO: 18, and a third polypeptide chain whose amino acid sequence is set forth in SEQ ID NO: 21; or
a first polypeptide chain whose amino acid sequence is set forth in SEQ ID NO: 22, and a second polypeptide chain whose amino acid sequence is set forth in SEQ ID NO: 23; or
a first polypeptide chain whose amino acid sequence is set forth in SEQ ID NO: 24, and a second polypeptide chain whose amino acid sequence is set forth in SEQ ID NO: 25; or
two heavy chains whose amino acid sequences are set forth in SEQ ID NO: 26, and two light chains whose amino acid sequences are set forth in SEQ ID NO: 18; or
two heavy chains whose amino acid sequences are set forth in SEQ ID NO: 27, and two light chains whose amino acid sequences are set forth in SEQ ID NO: 18; or
two heavy chains whose amino acid sequences are set forth in SEQ ID NO: 28, and two light chains whose amino acid sequences are set forth in SEQ ID NO: 18; or
two heavy chains whose amino acid sequences are set forth in SEQ ID NO: 29, and two light chains whose amino acid sequences are set forth in SEQ ID NO: 18.

15. A polynucleotide molecule whose nucleotide sequence is selected from:
(1) a nucleotide sequence encoding the bispecific antibody according to claim 1; and
(2) a complementary sequence of the nucleotide sequence of (1).

16. An expression vector comprising the polynucleotide molecule according to claim 15, wherein the expression vector is a eukaryotic expression vector.

17. A host cell comprising the polynucleotide molecule according to claim 15, wherein the host cell is a eukaryotic cell.

18. A method for preparing the bispecific antibody according to claim 1, wherein the method comprises culturing the host cell according to claim 17 under conditions suitable for the expression of the bispecific antibody to allow the host cell to express the bispecific antibody and collecting the expressed bispecific antibody from the host cell.

19. A pharmaceutical composition comprising the bispecific antibody according to claim 1, and a pharmaceutically acceptable carrier or excipient.

20. A method of preventing and/or treating cancer, comprising administering to a subject in need thereof, the pharmaceutical composition thereof according to claim 19, wherein the cancer is associated with CD112R and/or TIGIT.
